(19) United States
(12) Patent Application Publication
(10) Pub. No.: US 2009/0061524 A1
(43) Pub. Date: Mar. 5, 2009

(54) Enzyme-Channeling Based Electrochemical Biosensors

(76) Inventors: Judith Rishpon, Rechovot (IL); Tova Neufeld, Ariel (IL)

Correspondence Address: MARTIN D. MOYNIHAN d/b/a PRTSI, INC., P.O. BOX 16446, ARLINGTON, VA, 22215, US

(21) Appl. No.: 11/918,477

(22) PCT Filed: Apr. 11, 2006
(86) PCT No.: PCT/IL2006/000466

§ 371 Date: Oct. 15, 2007

Related U.S. Application Data
(60) Provisional application No. 60/671,482, filed on Apr. 15, 2005.

(51) Int. Cl. G01N 33/48; C12M 1/34

(52) U.S. Cl. 436/86; 435/287.2

(57) ABSTRACT

Low-cost, non-toxic and fast immunoassay systems (immunosensors) and uses thereof as analytical and diagnostic tools for detecting an immune response in a subject are disclosed. The systems and methods disclosed are based on recording an electrochemical signal which is generated proportionally to an enzymatic cascade reaction (enzyme-channeling) upon detecting an analyte, and therefore can be used to determine the titer level of an antibody analyte in a liquid sample such as artificial media, serum or blood both qualitatively and quantitatively, in a one-step and separation free immunoassay. Systems and methods based on recording an electrochemical signal which is generated proportionally to an enzymatic cascade reaction (enzyme-channeling) upon detecting an analyte, which utilize a non-toxic secondary substrate such as acetaminophen are also disclosed.

### FIELD AND BACKGROUND OF THE INVENTION

The present invention relates to electrochemical biosensors and, more particularly, to low-cost, separation-free and accurate electrochemical biosensors and uses thereof for qualitatively and quantitatively determining the presence of biological analytes such as antibodies in a liquid sample such as sera and blood.

Applications of different biosensors types for accurate measurements of chemicals, toxins and other analytes are in extensive use during the last decades. Electric, magnetic, electro-optic and piezoelectric are examples for commonly based sensor technologies.

Immunoassays have been widely used for the detection of antigens and antibodies. The most commonly used immunoassays are enzyme immunoassays (EIAs). The importance of EIAs, particularly in clinical analyses, medical diagnostics, pharmaceutical analyses, environmental control, food quality control, and bioprocess analyses, lies in their high sensitivity and specificity, which allow the detection of a wide spectrum of analytes in various sample matrices.

EIAs are commonly either heterogeneous (necessitating free antigen separation from those that have been bound to antibody) or homogeneous (requiring no separation or washing steps during the assay). Furthermore, EIAs can be either competitive or non-competitive, depending on the availability of antibody binding sites. Conventional EIAs are convenient for analysis of great numbers of samples on a routine basis and are widely used in a broad spectrum of applications. However, these methods require multiple washing and incubation steps to implement, and can be utilized in high volume only by complex and expensive analytical equipment. The need for multiple washing and incubation steps has also limited the development of portable point-of-care analytical devices that can be used to perform assays in decentralized locations.

In recent years, efforts have been made to overcome the limitations of heterogeneous EIAs and to search for homogeneous, rapid, and separation-free immunoassays that can be readily conducted at the point-of-care. Fast and simple EIA tests capable of detecting a single analyte with, for example, a color change that can be visually interpreted, have been developed. Based on the techniques of immobilizing antigen or antibody on a solid-phase support, assay formats such as dipsticks, test tubes, and wicking membrane test cartridges have been used to provide fast results for analytical conditions where a simple qualitative (yes/no) answer is clinically relevant. These membrane-based assays have gained increasing popularity in many areas of clinical chemistry. Not only that they form the basis of the majority of home use tests, but also are rapidly gaining use in the physician's office and hospital lab. These tests are widely accepted and increasingly used for detection of pregnancy, strep throat (an infection of the oral pharynx and tonsils by streptococcus), and bacteria, as well as for prediction of ovulation. Examples of such assays are described, for example, in U.S. Pat. Nos. 5,622,871, 4,703,017, 5,468,647, 5,622,871, and 5,798,273.

However, most of these rapid tests are incapable of performing sensitive and quantitative detection. As a result, medical diagnoses that require quantitative measurement of the target analyte remain within the domain of the complex immunoassay analyzers in centralized laboratories and similar facilities, requiring the use of multi-step, multi-reagent procedures, which are time and resource consuming tasks that typically require even several days to produce results.

The need for rapid, on-the-spot accurate tests for an immediate answer at the clinic and by the patient's bedside are of great importance. To date this filed is characterized by insufficient, partial and semi-quantitative solutions.

In recent years, intensive research has been undertaken to develop such diagnostic procedures that can be performed in the physician's office as well as in emergency ward. In this respect, amperometric-based measurement system can provide an attractive solution since it combines the high sensitivity and the relative simplicity of electrochemical techniques.

Thus, recent developments of rapid immunoassays had moved toward quantitative testing. The use of membrane-based immunoassays has been proposed for quantitative measurement of analytes. For example, U.S. Pat. No. 5,753,517 describes a quantitative immuno-chromatographic assay utilizing antibody-coated particles, independent control particles, and capillary flow through a membrane. However, there are difficulties in developing such quantitative immunoassays based on membrane format for point-of-care diagnostic tests. The most significant drawback of using membrane-based immunoassays arises from contradictory requirements from the solid supporting membrane. For example, immobilization of proteins in the detection area requires that the membrane have a strong binding affinity for the protein, but transport of analyte and particles containing detection components demands that the membrane would not bind to proteins. Furthermore, factors commonly used for increasing the performance of the membrane assay are often mutually exclusive, such as, for example, blocking reagents that reduce nonspecific interactions usually also reduce the amount of specific signal. In light of these competing requirements it becomes clear that conventional membrane systems are limited for use in quantitative and reliable immunoassays.

Immunoassays employing amperometric electrochemical detection have been applied to the determination of analytes in fluid samples. An immunoassay device using amperometric detection to perform diagnostic tests for analytes in body fluids is described, as a specific example, in U.S. Pat. Nos. 5,830,680 and 5,981,203. The device includes an electrochemical detection system for a separation-free sandwich-type immunoassay. Although such a device offers a separation-free feature, the time required for manipulating and incubating the sample limits the use of such assays for rapid diagnostic testing.

Other amperometric immunoassays employing the immobilization of antigens and antibodies, as well as electrochemical signal generating enzymes, on a multi-layered gel structure which coat the electrodes, are taught in U.S. Pat. Nos. 5,723,345 and 6,218,134. In these patents, analysis involves diffusion of the reagents through the layered gel. Yet, these techniques are particularly complicated to practice, are labor intensive and very expensive to implement, thus are not suitable for on-the-spot, disposable test-kits which can be utilized away from centralized laboratories.

Other immunoassays using electrochemical detection have to rely on methods conventional in heterogeneous immunoassays, such as lengthy incubation time and multiple washing steps to separate free antigen and detection reagent from bound ones. Yet, the limiting factor in the development of rapid separation-free electrochemical immunosensors remains the need of intensive time-consuming wash steps to avoid measuring the unbound enzyme label.

The concept of enzyme-channeling was first introduced to the field of immunosensors by Litman and Gibbons [Litman et al., Analytical biochemistry, 1980. 106(1): p. 223-229; and Gibbons, I., et al. in Methods in enzymology, 1987. 136: p. 93-103]. Using cascade reactions for signal generation, the enzyme label is linked catalytically to a second enzyme, which increases the sensitivity of the assay, and further gains higher efficiency when the cascade reactions (channeling) are carried out on the surface of a working electrode of an electrochemical sensor. Such signal generation mechanisms generated by a set of two co-enzymes can increase the signal by several orders of magnitudes, and hence are quite suitable for heterogeneous immunoassays which typically deal with a low signal-to-noise ratio.

Enzyme-channeling on the surface of a working electrode opened the route to development of one-step separation-free immunoassay amperometric immunosensors, [see, Rishpon, J. and D. Ivnitski, Biosensors & Bioelectronics, 1997. 12(3): p. 195-204; Ivnitski, D. and J. Rishpon, 1996. 11(4): p. 409-417; Ivnitski, D., et al., Bioelectrochemistry and Bioenergetics, 1998. 45(1): p. 27-32; Keay, R. W. and C. J. McNeil, Biosensors & Bioelectronics, 1998. 13(9): p. 963-970; and Wright, J. D., et al., Biosensors & Bioelectronics, 1995. 10(5): p. 495-500]. These techniques were developed so as to be implemented by using simple and disposable graphite electrodes and standard protein immobilization techniques which are well established in the art.

The immunoassays taught by Prof. Rishpon, a co-inventor of the present invention, and co-workers, [Rishpon, J. and D. Ivnitski, Biosensors & Bioelectronics, 1997. 12(3): p. 195-204; Ivnitski, D. and J. Rishpon, 1996. 11(4): p. 409-417; Ivnitski, D., et al., Bioelectrochemistry and Bioenergetics, 1998. 45(1): p. 27-32;] effected by an enzyme-channeling system, employed the availability of a co-enzymes pair (CE1 and CE2), an affinity-purified antibody, namely an IgG molecule of a specific animal (acting as an analyte), an affinity-purified antisera (antibodies) against that entire IgG molecule (αIgG), and a conjugate of the antisera and one of the co-enzymes of the enzyme-channeling system (αIgG-CE2). According to these teachings, the IgG or the αIgG was immobilized on the surface of the working electrode, by means of a polymer and a cross-linking agent, together with the other co-enzymes of the enzyme-channeling system (CE1). In one of the immunoassays, according to these teachings, the analyte (IgG) is detected by the principle of a sandwich-type assay wherein the IgG binds to the immobilized αIgG on one side, and an αIgG-CE2 conjugate binds to the IgG on the other side, thus bringing the two co-enzymes into close proximity. This proximity enables the generation of a strong signal. Further according to these teachings, the analyte (IgG) can be quantitatively detected by displacement thereof from an immobilized αIgG which is effected by competitive binding of standard samples of the analyte conjugated to the CE2 (IgG-CE2) while monitoring the reduction of the signal.

Still, these teaching are limited in that the signal-generating enzymatic reaction required the use of redox-prone secondary substrates, which by nature are oftentimes toxic and/or unstable, such as p-phenylene diamine dihydrochloride or potassium iodide. This limitation prohibits mass production of user- and environmentally-friendly enzyme-channeling-based diagnostic kits.

Moreover, an immunosensor which is based on immobilizing an antibody for detecting the corresponding antigen in a given sample requires that a set of antigen-specific antibodies, or an antigen-specific monoclonal antibody, is identified, produced, isolated and handled, namely immobilized on an electrode. The identification and affinity-based isolation of an antigen-specific set of antibodies is a time consuming process, and producing a subset of monoclonal antibodies adds significantly high-cost and lengthy procedures. Furthermore, in the more common case where the immunoassay is designed to detect the level of an immune response towards a pathogenic microorganism, an antibody-based immunoassay will be highly sensitive to each mutation in the antigen. Such mutations in the antigen may be frequent, and may disrupt the binding of all or some of the antibodies which were produced for the pre-mutated form of the antigen of a given microorganism. Thus, even a minute mutation in the antigen may alter some or even all the epitopes, hence rendering the antibodies which were produce for that pre-mutated antigen obsolete or ineffective, and subsequently rendering the immunoassay system valueless.

Furthermore, as in the case of most complex biological macromolecules, antibodies oftentimes lose activity due to experimental and storage conditions and due to the immobilization process. Therefore many technical and practical problems arise from the fact that antibodies are complex and delicate proteins.

These limitations prohibit mass production of low-cost and user- and environmentally-friendly disposable immunoassay-based diagnostic kits.

There is thus a widely recognized need for, and it would be highly advantageous to have, one-step and separation-free immunoassay-based diagnostic methods which can be implemented in fast, sensitive, accurately quantitative and low-cost devices, devoid of the above limitations.

### SUMMARY OF THE INVENTION

The present invention is of novel immunoassay systems (immunosensor) which are based on recording an electrochemical signal which is generated proportionally to an enzymatic cascade (enzyme-channeling), upon detecting an analyte, and which include an antigen immobilized to a working electrode in the system and hence can be used to determine the titer level of an antibody analyte in a liquid sample such as artificial media, serum or blood both qualitatively and quantitatively, serving as an efficient analytical and diagnostic tool for detecting an immune response in a subject. The present invention is further of similar, enzyme-channeling based bioassay systems (biosensors), in which a secondary substrate of at least one of the enzymes in the enzymatic cascade is the non-toxic acetaminophen, and hence these systems can be efficiently utilized for detecting various analytes that form a part of a binding pair, such as antibodies, antigens, receptors, ligands, enzymes, inhibitors and the like.

Thus, according to one aspect of the present invention there is provided a system for detecting an antibody in a liquid sample, the system includes an electrochemical cell which includes a reference electrode, a counter electrode, an electrolytic solution, a current detecting unit and a working electrode having immobilized thereon an antigen and a first enzyme of an enzymatic cascade. The system further includes a conjugate which comprises of an agent capable of specifically binding to the antibody and a second enzyme of the enzymatic cascade being conjugated to the agent and a substrate of the first enzyme of the enzymatic cascade, wherein the antigen is capable of specifically binding to the antibody and the first enzyme is capable of catalyzing the formation of a substrate of the second enzyme, and further wherein the second enzyme generates an electrochemically detectable moiety upon binding of the conjugate to the antibody and binding of the antibody to the antigen, whereas a presence and/or amount of the electrochemically detectable moiety is detectable by the detecting unit.

According to features in preferred embodiments of the invention described below, the system further includes a secondary substrate of the second enzyme.

According to another aspect of the present invention there is provided a kit for detecting an antibody in a liquid sample, the kit includes a working electrode having immobilized thereon an antigen and a first enzyme of an enzymatic cascade as presented herein.

According to further features in the described preferred embodiments, the kit further includes a conjugate as presented herein.

According to still further features in the described preferred embodiments, the kit further includes a substrate of the first enzyme.

According to still further features in the described preferred embodiments, the kit further includes a secondary substrate of the second enzyme.

According to yet further features in the described preferred embodiments, the kit further includes at least one of a reference electrode, a counter electrode, an electrolytic solution and a current detecting unit.

According to yet further features in the described preferred embodiments, the kit further includes a conjugate as presented herein and/or a substrate of the first enzyme and/or a secondary substrate of the second enzyme and/or at least one of a reference electrode, a counter electrode, an electrolytic solution and a current detecting unit.

According to yet another aspect of the present invention there is provided a method of detecting an antibody in a liquid sample, the method includes contacting the liquid sample with a system as presented herein, applying a pre-selected potential between the working electrode and the counter electrode, recording a current formed between the working electrode and the counter electrode and determining the presence and/or amount of the electrochemically detectable moiety, thereby detecting the antibody in the liquid sample.

According to features in preferred embodiments of the invention described below, contacting the liquid sample with the system includes adding the liquid sample and the conjugate to the electrochemical cell, and subsequently adding to the cell the substrate of the first enzyme, to thereby initiate the enzymatic cascade.

According to further features in preferred embodiments, adding the liquid sample and adding the conjugate to the electrochemical cell is performed concomitantly.

According to further features in preferred embodiments, adding the liquid sample and adding the conjugate to the electrochemical cell is performed sequentially.

According to still further features in preferred embodiments, the system further includes a secondary substrate of the second enzyme.

According to features in preferred embodiments of the invention described below, contacting the liquid sample with the system having a secondary substrate includes adding the liquid sample and the conjugate to the electrochemical cell, adding the secondary substrate to the electrochemical cell and subsequently adding to the cell the substrate of the first enzyme.

According to features in preferred embodiments, adding the liquid sample and the conjugate to the electrochemical cell is performed concomitantly.

According to further features in preferred embodiments, adding the liquid sample, the conjugate and the secondary substrate to the electrochemical cell is performed concomitantly.

According to yet further features in preferred embodiments, adding the liquid sample, the conjugate and the secondary substrate to the electrochemical cell is performed sequentially.

According to yet further features in preferred embodiments, adding the liquid sample, the conjugate and the secondary substrate to the electrochemical cell is performed concomitantly and adding the substrate of the first enzyme is performed subsequent to adding the liquid sample and the conjugate.

According to yet another aspect of the present invention there is provided a system for detecting a first member of a binding pair in a liquid sample, the system includes an electrochemical cell having a reference electrode, a counter electrode, an electrolytic solution, a current detecting unit and a working electrode having immobilized thereon a second member of the binding pair and a first enzyme of an enzymatic cascade. The system further includes a conjugate which comprises an agent capable of specifically binding to the first member of the binding pair and a second enzyme of the enzymatic cascade conjugated to the agent, a substrate of the first enzyme of the enzymatic cascade and a secondary substrate of the second enzyme of the enzymatic cascade. The system is characterized by having the first enzyme of the enzymatic cascade which is a hydrogen peroxide-producing enzyme, the second enzyme of the enzymatic cascade being a peroxidase and the secondary substrate being acetaminophen, and further wherein the second enzyme generates a detectable form of the acetaminophen upon binding of the conjugate to the first member of the binding pair and binding of the first member to the second member of the binding pair, whereas a presence and/or amount of the detectable form of the acetaminophen is detectable by the detecting unit.

According to further features in preferred embodiments of the invention described below, the binding pair is selected from the group consisting of a receptor—ligand binding pair, an enzyme—inhibitor binding pair, an enzyme—substrate binding pair, polynucleotide sequence—complimentary polynucleotide sequence binding pair and an antigen—antibody binding pair.

According to still another aspect of the present invention there is provided a method of detecting a first member of a binding pair in a liquid sample, the method which includes contacting the liquid sample with a system as presented herein, applying a pre-selected potential between the working electrode and the counter electrode, recording a current formed between the working electrode and the counter electrode and determining the presence and/or amount of the detectable form of the acetaminophen, thereby detecting the first member of a binding pair in the liquid sample.

According to features in preferred embodiments of the invention described below, contacting the system with the liquid sample includes adding the liquid sample and the conjugate to the electrochemical cell, adding the acetaminophen to the electrochemical cell, and subsequently adding to the cell the substrate of the first enzyme.

According to further features in preferred embodiments, adding the liquid sample and the conjugate to the electrochemical cell is performed concomitantly.

According to further features in preferred embodiments, adding the liquid sample, the conjugate and the acetaminophen to the electrochemical cell is performed concomitantly.

According to further features in preferred embodiments, adding the liquid sample, the conjugate and the acetaminophen to the electrochemical cell is performed sequentially.

According to further features in preferred embodiments, adding the liquid sample, the conjugate and the acetaminophen to the electrochemical cell is performed concomitantly and adding the substrate of the first enzyme is performed subsequent to adding the liquid sample and the conjugate.

According to still another aspect of the present invention, there is provided an electrode for detecting an antibody in a liquid sample, the electrode includes a body and a surface having immobilized thereon an antigen and a first enzyme of an enzymatic cascade, the antigen is capable of specifically binding to the antibody, the first enzyme is capable of catalyzing a formation of a substrate of a second enzyme in the enzymatic cascade, the second enzyme capable of generating an electrochemically detectable moiety upon binding of a conjugate to the antibody and binding of the antibody to the antigen, whereby the conjugate comprises an agent capable of specifically binding to the antibody and the second enzyme of the enzymatic cascade being conjugated to the agent.

According to features in preferred embodiments of the invention described below, the working electrode's body is made of a conductive material which is selected from the group consisting of graphite, carbon ink, gold, platinum, silver, copper, nickel, chromium, and palladium. Preferably, the conductive material is selected from the group consisting of graphite and carbon ink, thus preferably the working electrode is selected from the group consisting of a graphite electrode, a carbon ink electrode and a screen printed electrode.

According to features in preferred embodiments of the invention described below, the working electrode or a surface thereof, further includes an immobilization layer applied thereon.

According to features in preferred embodiments, the antigen or the first member of a binding pair, and the first enzyme of an enzymatic cascade are immobilized on the working electrode via the immobilization layer.

According to further features in preferred embodiments, the immobilization layer includes a polymer attached to the surface of the working electrode and a cross-linking agent attached to the polymer.

According to still further features in preferred embodiments, the polymer is selected from the group consisting of polyethyleneimine, chitosan, polyethylene oxide, polyvinylalcohol, polyvinyl acetate, polyacrylamide, poly(vinylpyrrolidone), poly(2-vinylpyridine), poly(4-vinylpyridine), poly(4-vinyl-N-butylpyridinium) bromide and poly(vinylbenzyltrimethyl)ammonium hydroxide. Preferably the polymer is polyethyleneimine.

According to still further features in preferred embodiments, the cross-linking agent is selected from the group consisting of glutaraldehyde, polyglutaraldehyde, bis(imido ester), bis(succinimidyl ester), diisocyanate, succinimidyl acetylthioacetate, hydrazine, succinimidyl 3-(2-pyridyldithio)propionate, 3-(2-pyridyldithio)propionyl and tris-(2-carboxyethyl)phosphine. Preferably the cross-linking agent is polyglutaraldehyde.

According to features in preferred embodiments of the invention described below, the antigen and the first enzyme are attached to the cross-linking agent.

According to features in preferred embodiments of the invention described below, the immobilization layer includes a microporous membrane.

According to features in preferred embodiments of the invention described below, the antigen and the first enzyme are attached to the microporous membrane.

According to further features in preferred embodiments, the microporous membrane is at least permeable at least to the electrochemically detectable moiety.

According to further features in preferred embodiments, contacting the sample with the working electrode further includes washing the electrochemical cell upon adding the liquid sample and/or upon adding the conjugate.

According to yet further features in preferred embodiments of the present invention as presented hereinbelow, contacting the sample with the working electrode is effected without washing the cell.

According to further features in preferred embodiments, the molar ratio of the conjugate and the antigen ranges from about 1:100 to about 1:10,000, preferably the molar ratio ranges from about 1:100 to about 1:5,000, and most preferably the molar ratio is about 1:1000.

According to features in preferred embodiments of the invention described below, the electrochemically detectable moiety is generated in proximity to the working electrode.

According to features in preferred embodiments of the invention described below, the antigen is not an antibody.

According to features in preferred embodiments of the invention described below, the detection of the antibody or the second member of a bind pair is qualitative.

According to features in preferred embodiments of the invention described below, the detection of the antibody or the second member of a bind pair is quantitative.

According to further features in preferred embodiments of the invention described below, the molar ratio between the antigen or the first member of a binding pair and the first enzyme ranges from about 1:5 to about 5:1, more preferably the molar ratio ranges from about 1:2 to about 2:1, and most preferably the molar ratio is about 1:1.

According to features in preferred embodiments of the invention described below, the first enzyme is a hydrogen peroxide producing enzyme, and preferably the first enzyme is glucose oxidase.

According to further features in preferred embodiments of the invention described below, the second enzyme is a peroxidase, and preferably the second enzyme is horseradish peroxidase.

According to still further features in preferred embodiments of the invention described below, the secondary substrate is selected from the group consisting of potassium iodide (KI), p-phenylene diamine dihydrochloride (PPD) and acetaminophen, and preferably it is acetaminophen.

According to yet further features in preferred embodiments of the invention described below, the agent capable of specifically binding to the antibody is an antiserum antibody.

According to features in preferred embodiments of the invention described below, the systems, kits, electrode and methods presented herein are being for detecting an immune response.

According to features in preferred embodiments, the immune response is selected from the group consisting of an immune response to a pathogenic microorganism, an immune response to a toxin, an immune response to a drug, an immune response to a foreign particle, an immune response to an organ transplant and an immune response to an implant. Preferably, the pathogenic microorganism is a canine pathogen, and most preferably the canine pathogen is a canine distemper virus. The present invention successfully addresses the shortcomings of the presently known configurations by providing novel immunoassay systems and methods of using the same, which can detect an antibody in a liquid sample in a separation-free and fast mode, both qualitatively and quantitatively.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of an exemplary system according to the present invention wherein glucose oxidase (GOX), serving as the first enzyme of the enzymatic cascade, and an antigen are attached to an immobilization layer (marked by a wavy line) which coats the working electrode, and wherein glucose, serving as the substrate of the first enzyme, is converted to gluconolactone and hydrogen peroxide, which serves as the substrate of the second enzyme, horseradish peroxidase (HRP), and wherein the conjugate is an antisera antigen attached to HRP, and wherein HRP generates the electrochemically detectible moiety from a secondary substrate;

FIG. 2 is a schematic illustration of an exemplary system according to the present invention wherein glucose oxidase (GOX), serving as the first enzyme of the enzymatic cascade, and an antigen are attached to a membrane serving as an immobilization layer (marked by a heavy dashed line) which is laid on the working electrode, and wherein glucose, serving as the substrate of the first enzyme, is converted to gluconolactone and hydrogen peroxide, which serves as the substrate of the second enzyme, horseradish peroxidase (HRP), and wherein the conjugate is an antisera antigen attached to HRP, and wherein HRP generates an electrochemically detectible moiety from a secondary substrate;

FIG. 3 presents a schematic illustration of an electrochemical cell fitted with screen-printed counter and reference electrodes and a graphite working electrode connected to the rotating device, onto which an antigen and an enzyme of the enzyme-channeling dyad are attached, connected to a central control and a signal recording and processing unit;

FIGS. 4a-c present a schematic illustration of membrane-based electrochemical cell comprising three screen-printed electrodes (SPEs); a working electrode in the center, surrounded by a crescent-shaped counter electrode and a dot-shaped reference electrode printed with carbon ink on an insulating plate (a), and further showing a membrane onto which an antigen and an enzyme of the enzyme-channeling dyad are attached, laid on-top of the screen-printed electrodes (b) and a cylinder constituting the electrochemical reaction vessel, placed on-top of the membrane (c);

FIG. 5 is a comparative bar graph, presenting the maximal signals recorded with an exemplary system according to the present embodiments, using a membrane-based immunoassays systems wherein the antigen, CDV, and the enzyme, GOX, are immobilized thereon at two relative ratios of 1:1 GOX to CDV (denoted 1:1 Ag) and 1:10 GOX to CDV (denoted 1:10 Ag), α-dog-IgG-HRP as the conjugate, acetaminophen as a secondary substrate for HRP and glucose as the substrate for GOX, designed to detect antibodies against canine distemper virus in a sample of dog serum denoted “positive serum770(1:1 Ag)” and marked by a black bar, a sample of dog serum denoted “positive serum770(1:10 Ag)” and marked by a red bar, a sample of dog serum denoted “low level serum (1:1)CPDV” and marked by a light green bar, a sample of dog serum denoted “low level serum (1:10)CPDV” and marked by a yellow bar, a sample of dog serum denoted “negative serum#4(1:1Ag)” and marked by a blue bar, a sample of dog serum denoted “negative serum#4(1:1Ag)” and marked by a magenta bar, a sample of dog serum denoted “negative serum#4(1:10Ag)” and marked by a cyan bar, a sample of dog serum denoted “negative serum#4(1:10Ag)” and marked by a gray bar, a control sample denoted “no serum(1:1Ag)” and marked by a brown bar, a control sample denoted “no serum(1:1Ag)” and marked by a dark green bar, a control sample denoted “no serum(1:10Ag)” and marked by a olive bar, and a control sample denoted “no serum(1:10Ag)” and marked by a navy blue bar;

FIG. 6 presents comparative plots presenting the electrochemical signal response as recorded over time in a separation-free immunoassay with an exemplary system according to the present embodiments, using a graphite working electrode having GOX and dog-IgG or BSA immobilized thereon, PPD as HRP secondary substrate, α-dog-IgG-HRP as the conjugate and glucose as the substrate for GOX, wherein glucose, PPD, and the conjugate were added successively, showing that the recorded signals are not notable upon the addition of the substrates (as marked by the left arrow), but are notable after the addition of the conjugate (as marked by the right arrow);

FIGS. 7a-b are comparative plots showing the electrochemical signal response as recorded over time in a separation-free immunoassay with an exemplary system according to the present embodiments, using a graphite working electrode having GOX and dog-IgG or BSA immobilized thereon, acetaminophen (AAP) as HRP secondary substrate, α-dog-IgG-HRP as the conjugate and glucose as the substrate for GOX, wherein glucose, AAP and the conjugate are added successively in that order, showing that the addition of AAP and glucose did not affect the signal, and further showing that the difference between the tests conducted with immobilized dog-IgG (duplicate tests in green and black curves in FIG. 7a and triplicate tests in green, blue and black curves in FIG. 7b) and the control tests conducted with immobilized BSA (duplicate tests red and yellow curves in FIGS. 7a and 7b) was noted only upon addition of the α-dog-IgG-HRP conjugate, and yet further showing the improvement of the signal-to-noise ratio of the experiments upon the addition of Tween-20 to the reaction cell, which reduces the non-specific signals recorded without Tween-20 for the BSA-loaded electrodes (duplicate tests red and yellow curves in FIG. 7a) as compared to the signals recorded with Tween-20 (duplicate tests red and yellow curves in FIG. 7b);

FIGS. 8a-b are comparative plots showing the electrochemical signal response as recorded over time in a one-step and separation-free immunoassay with an exemplary system according to the present embodiments, using a graphite working electrode having GOX and dog-IgG or BSA immobilized thereon at two relative ratios of 1:1 of GOX to dog-IgG and 1:2 of GOX to dog-IgG, acetaminophen (AAP) as HRP secondary substrate, α-dog-IgG-HRP as the conjugate and glucose as the substrate for GOX, wherein the enzyme substrates and the conjugate are added concomitantly in one-step, showing the elimination of the none-specific interactions (black curves in FIGS. 8a and 8b) thus demonstrating the substantial improvement of the one-step and separation-free approach, and further showing the improvement of the signal-to-noise ratio between the signals recorded using the GOX/dog-IgG electrodes (red curves in FIGS. 8a and 8b) or the control GOX/BSA electrodes (black curves in FIGS. 8a and 8b) prepared with a 1:1 ratio of GOX to dog-IgG (a), as compared to the curves recorded using electrodes prepared with a 1:2 ratio of GOX to dog-IgG (b);

FIGS. 9a-b are comparative plots and a bar graph presenting the electrochemical signal obtained in a one-step and separation-free immunoassay with an avidin-biotin model system, using a graphite working electrode having GOX and avidin or GOX and BSA immobilized thereon, acetaminophen (AAP) as HRP secondary substrate, biotin-HRP as the conjugate and glucose as the substrate for GOX, showing that the electrochemical signal as recorded over time (a) produced a notable signals (red curve in FIG. 9a) whereby the BSA control experiment showed no signal (blue curve in FIG. 9a), as was reproduced three times using three different detection systems (b), thereby validating the concept of enzyme channeling in the context of one-step and separation-free immunoassays;

FIG. 10 presents comparative plots presenting the electrochemical signal response as recorded over time in a separation-free immunoassay with an exemplary system according to the present embodiments, using screen printed working electrodes having GOX and avidin or GOX and BSA immobilized thereon, acetaminophen (AAP) as HRP secondary substrate, biotin-HRP as the conjugate and glucose as the substrate for GOX, showing notable signals produced by two repeating experiments using an avidin-loaded working SPE (black and blue curves) are systematic and reproducible and exhibited high specificity as compared to the two repeating control experiments using an BSA-loaded working SPE (red and yellow curves);

FIG. 11 presents comparative plots of the electrochemical signal response as recorded over time in a non-separation-free and non-enzyme-channeled immunoassay with an exemplary system according to the present embodiments, using membrane-based working electrodes having canine distemper antigen (CDV) immobilized thereon, hydrogen peroxide as the substrate for HRP, acetaminophen (AAP) as HRP secondary substrate and α-dog-IgG-HRP as the conjugate, showing a clear difference between the notable signal for dog serum samples positive for CDV (repeating black and blue curves) and the negligible signal for dog serum samples negative for CDV (repeating red and yellow curves) as recorded upon the addition of hydrogen-peroxide to the reaction cell (marked by two black arrows, one for each repeat), thereby demonstrating the reliability of the immunoassay concept presented herein using a membrane and SPEs;

FIG. 12 presents comparative plots of the electrochemical signal response as recorded over time in a one-step separation-free immunoassay with an exemplary system according to the present embodiments, using a screen printed working electrode having canine distemper virus (CDV) antigen and GOX immobilized thereon, acetaminophen (AAP) as HRP secondary substrate, α-dog-IgG-HRP as the conjugate and glucose as the substrate for GOX, showing notable signal produced for positive dog serum sample diluted 1:100 (magenta curve) and a weak signal produced for negative dog serum (SPF) sample diluted 1:100 (black curve), showing a clear difference between the positive and negative sera, thus demonstrating the reliability of the immunoassay concept presented herein using a SPE for a working electrode;

FIG. 13 presents a comparative plots of the electrochemical signal response as recorded over time in a one-step, separation-free and sandwich immunoassay with an exemplary system according to the present embodiments, using a screen printed working electrode having avidin and GOX immobilized thereon, a biotin-CDV conjugate for binding antibodies against CDV in the samples, acetaminophen as HRP secondary substrate, glucose as GOX substrate and an α-dog-IgG-HRP conjugate, showing a clear difference between the notable signal recorded for positive dog serum samples (black, blue and magenta repeating curves) as compared to the weaker signal recorded for negative (SPF) dog serum samples (red and yellow repeating curves), thus demonstrating the reliability of the immunoassay concept presented herein using a SPE for a working electrode;

FIG. 14 presents comparative plots of the electrochemical signal response as recorded over time in a one-step, separation-free and sandwich immunoassay with an exemplary system according to the present embodiments, using a screen printed working electrode having avidin and GOX immobilized thereon, a biotin-CDV conjugate for binding antibodies against CDV in the samples, acetaminophen as HRP secondary substrate, glucose as GOX substrate and an α-dog-IgG-HRP conjugate, showing a notable signal recorded for a sample of dog sera strongly positive for CDV denoted “strong positive” and marked by a red curve, a sample of dog sera negative for CDV denoted “SPF” and marked by a yellow curve, a sample of dog sera moderately positive for CDV denoted “serum 8” and marked by a black curve, and a sample of dog sera moderately positive for CDV denoted “serum poly” and marked by a blue curve, showing a high correlation between the antibody titer level in the samples and the recorded signals thereof; and

FIGS. 15a-b are two comparative bar diagrams, presenting the maximal signals recorded with an exemplary system according to the present embodiments, using the membrane-based immunoassay system described in FIG. 14 hereinabove and shown in FIG. 15a, and the maximal signals recorded with the commercial ImmunoComb analytical system shown in FIG. 15b, showing a high correlation between the advantageous one-step, separation-free and enzyme-channeling based immunoassay system and the disadvantageous non-one-step, non-separation-free and non-enzyme-channeling based commercial system.

### DESCRIPTION OF THE PREFERRED EMBODIMENTS

The present invention is of novel immunoassay systems (immunosensors) which are based on recording an electrochemical signal which is generated proportionally to an enzymatic cascade (enzyme-channeling), upon detecting an analyte, and which include an antigen immobilized to a working electrode in the system and hence can be used to determine the titer level of an antibody analyte in a liquid sample such as serum or blood both qualitatively and quantitatively, serving as an efficient analytical and diagnostic tool for detecting an immune response in a subject. The present invention is further of similar, enzyme-channeling based bioassay systems (biosensors), in which a secondary substrate of at least one of the enzymes in the enzymatic cascade is the non-toxic acetaminophen, and hence these systems can be efficiently utilized for detecting various analytes that form a part of a binding pair, such as antibodies, antigens, receptors, ligands, enzymes, inhibitors and the like.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, current systems and methods for detecting, both qualitatively and quantitatively, the titer level of antibodies in a liquid sample suffer from great limitations such as complexity and costliness of the systems, and lengthy, cumbersome and inconvenient experimental protocols which are typically performed in central laboratories. Even the most commonly used enzyme-linked immunosorbent assay (ELISA) systems require lengthy wash steps in specialized machines and special readers for analysis.

These limitations stem mainly from the fact that practical and useful immune response diagnostics must be sufficiently sensitive so as to detect low levels of a desired analyte, e.g., an antibody, in a heterogeneous sample containing many other compounds and antibodies. Moreover, typically, the binding event between the analyzer, e.g., one member of a binding pair such as an antibody-antigen pair, and the analyte, namely the other member of a binding pair, is hardly detectable since it often produces no detectable signal. Systems which attempt to record and measure these events must be based on secondary events which can be coupled proportionally to the aforementioned binding event. This coupling renders these systems even more complex and less accurate, and in some cases involves the use of harmful (e.g., toxic) chemicals and reagents.

As mentioned above, employing the concept of an enzyme-channeling reaction, coupled to the immuno-binding event, to produce an electrochemically detectable signal by producing an electrochemically detectable moiety as a result of an enzymatic cascade which occurs upon occurrence of the immuno-binding event, has been proposed by several researchers in the passed decade, a co-inventor of the present invention. The coupling of an enzyme channeling mechanism to the immunoassay eliminates the need for extensive wash steps; hence, a separation free process is made possible due to the electron transfer mediated signal which is generated mainly or only when the two enzymes are brought into close proximity upon binding of the immunological components of the immunoassay. The presently known systems require, for example, the immobilization of an antibody or avidin to an electrode, together with the immobilization of an enzyme of the enzymatic cascade, such as glucose oxidase. Further, these systems require the use of a conjugate comprising an antigen or an antiserum antigen or biotin-labeled counterpart thereof, linked to the second enzyme of the enzymatic cascade.

Although proven in principle, these systems still suffer from inaccuracy and low signal-to-noise ratio, mainly due to non-specific interactions near the electrode that could be alleviated only by intensive wash-steps which inevitably defeated the objective of having a separation-free and fast immunoassay system.

While conceiving the present invention, the present inventors considered that since one antigen brings about the production of many antibodies which would all bind to it specifically; each per one epitope, this one-to-many ratio can be harnessed in favor of the immuno-binding event required in an immunoassay system. It was hypothesized by the present inventors that by immobilizing the antigen rather than the antibody to the electrode, the one-to-many ratio would favor specific interaction near the electrode and thus improve the sensitivity of an enzyme-channeling based immunoassay system.

Moreover, antigen-antibody binding requires the structure of the globular antibody, which might be affected upon antibody immobilization. Hence, the present inventors considered that immobilizing the antigen rather than the antibody would alleviate many problems which arise from the fact that antibodies are complex and delicate proteins which oftentimes lose activity due to the immobilization process, and further require special handling and conditions, even when immobilized on an element which forms a part of a diagnostic kit, which are not always possible in on-spot diagnosis situations. Furthermore, antibody-based electrodes typically require the use of monoclonal antibodies which are an expensive and hard to produce component of the system.

In contrast, antigens, which can be selected so as to be minimal in size and complexity while still retaining many of their epitopes, are more stable. Some antigens are small haptens, short peptides and polysaccharides and combinations thereof which are far less sensitive than large proteins such as antibodies.

Moreover, antigens can be selected such that the immobilization process will have only a minimal or no effect on their three dimensional configuration, as can be effected, for example, with a linker moiety. Therefore, while parts of the antigen may still become inaccessible to some antibodies due to epitope hindrance which is caused by the immobilization, other epitopes will still be available for binding with other specific antibodies.

While reducing the present invention to practice, the present inventors have successfully practiced the immobilization of an antigen and an enzyme that forms a part of an enzyme cascade to a working electrode, and successfully used this electrode in a system for immunoassays, as demonstrated in the Example section that follows.

Hence, according to one aspect of the present invention, there is provided a system for detecting an antibody in a liquid sample, which comprises an electrochemical cell having components which are common to other similar systems, such as a reference electrode, a counter electrode, an electrolytic solution and a current detecting unit, as defined hereinbelow, and a working electrode having immobilized proximally thereon an antigen and a first enzyme of an enzymatic cascade. The system further comprises a conjugate of an agent capable of specifically binding to the antibody and a second enzyme of the enzymatic cascade being conjugated to this agent, and a substrate of the first enzyme of the enzymatic cascade.

As used herein throughout, the term “detecting” encompasses qualitatively and/or quantitatively determining the presence and/or level (e.g., concentration, concentration variations) of an analyte (e.g., an antibody) in the sample.

The phrase “liquid sample”, as used herein, refers to a solution of biological or artificial origins, or a sample of treated biological liquid, which comprises the antibody. A biological liquid may be any bodily fluid which comprises the antibody such as, for example, blood, serum, saliva and mucus. A liquid sample of artificial origins may be, for a non-limiting example, a culture medium which comprises in vitro produced antibodies, such as, for example, hybridoma conditioned medium.

The system presented herein is based on typical electrochemical systems known and used in the art, and includes electrodes placed in or on an insulating base or plate. The electrodes of a typical electrochemical system are made of conductive materials such as carbon or metal, and include a working electrode as presented herein, and a counter (also referred to as an auxiliary electrode) electrode. The electrode system can further include a reference electrode, such as, for example, a saturated calomel electrode.

As in typical electrochemical systems, when the liquid sample containing the analyte is placed in the electrochemical cell and brought in contact with the electrodes, and other components of the system presented herein are added thereto, the combination of the immunologic and enzymatic reactions produces a transfer of electrons (an electric current). The presence and magnitude of the electric current, which is proportional to the concentration of the analyte in the liquid sample, is recorded by the signal recording and processing unit which forms a part of the system presented herein.

As used herein, the term “antibody”, which is synonymous with the terms “immunoglobulin” or “Ig”, refers to a globular protein which is produced in special cells of the immune system in response to the presence in the body of antigen(s), and which is capable of binding to an antigen. The body's immune system includes hundreds of thousands of different white blood cells called B lymphocytes, each capable of producing one type of antibody and each bearing sites on its membrane that will bind with a specific antigen. When such a binding occurs, it triggers the B lymphocyte to reproduce itself, forming a clone that manufactures vast amounts of its antibody.

The antibody molecule is composed of four polypeptide chains; two identical light chains and two identical heavy chains, joined by disulfide bridges. The heavy chains are characterized by a unique sequence per native or mutant species, hence can be used as a finger-print antigenic feature across species.

The light chains have a variable portion that is different in each type of antibody and is the active portion of the molecule that binds with the specific antigen by recognizing a unique epitope. Antibodies combine with some antigens, such as bacterial toxins, and thus neutralize their effect; they remove other substances from circulation in body fluids; they bind certain antigens together, a process known as agglutination; and they activate complement, blood serum proteins that cause the destruction of the invading cells.

As used herein, the term “antibody” encompasses antibodies of any class of naturally occurring antibodies, such as, for example, IgG, IgG₁, IgG₂, IgG₂ₐ, IgG_{2b}, IgG_{2c}, IgG₃, IgG₄, IgM, IgE, IgA, IgA₁, IgA₁, IgA₁, IgY and IgD, synthetic antibodies which are not necessarily produced by an immune system, and a substantially intact antibody molecule or a functional fragment thereof that is capable of binding to an antigen. Suitable antibody fragments for practicing the present invention include, inter alia, a complementarity-determining region (CDR) of an immunoglobulin light chain, a CDR of an immunoglobulin heavy chain, a variable region of a light chain, a variable region of a heavy chain, a light chain, a heavy chain, an Fd fragment, and antibody fragments comprising essentially whole variable regions of both light and heavy chains such as an Fv, a single-chain Fv, an Fab, an Fab′, and an F(ab′)2.

Antibodies may be developed, naturally or synthetically, against other antibodies. For example, an anti-dog antibody, or α-dog-IgG, is an antibody which will recognize and bind all antibodies which are produce in dogs of all sub-species.

The term “antigen” as used herein, refers to a substance that when introduced into the body stimulates the production of an antibody. Antigens include toxins, bacteria, viruses, and any type of foreign cells including blood cells and cells of transplanted organs. Antigens are identified as foreign by the body's immune system, triggering the release of antibodies as part of the body's immune response. Antigens are typically proteins, polysaccharides or combinations thereof, but can also be any type of molecule, including small molecules (haptens), typically coupled to a carrier-protein. An antigen-antibody binding pair, is typically characterized by a binding affinity, also referred to as a dissociation constant (K_{D}), of at least 10⁻⁵ M. While antigens can sometimes be antibodies, preferably, the antigens utilized in this context of the present invention are not antibodies.

The term “hapten”, as used herein, refers to a small molecule which can elicit an immune response only when attached to a large carrier such as a protein; the carrier may be one which also does not elicit an immune response by itself. Once the body has generated antibodies to a hapten-carrier adduct, the small-molecule hapten will typically not initiate an immune response by itself, but will be able to bind to the antibody. By having a small size, a hapten typically have fewer epitopes as compared to other antigens.

The term “epitope”, as used herein, is synonymous with the phrase “antigenic determinant” and refers to a specific chemical domain, a unique molecular shape or a molecular region which exists on an antigen's surface and is sufficient for antibody production and therefore antibody binding. The epitope stimulates the production of, and is recognized by a specific and unique antibody or T-cell receptor; hence, each epitope on a molecule, such as a regional amino-acid sequence of a protein, elicits the synthesis of a different antibody.

Therefore, the immobilized antigen is selected capable of specifically binding to the antibody, which is the analyte in question.

The system, according to the present invention, further includes an agent capable of specifically binding to the antibody, which is conjugated to the second enzyme. The role of this agent is to physically and chemically couple the enzymatic cascade event with the immuno-binding event, such that these two events will occur in close proximity. In general, any agent which can bind specifically to the antibody (the analyte in question) in a different recognition mode than the antigen, so as not to compete with the antigen-antibody interaction, such as another antibody against the analyte, or other antibody-binding factors such as, for example, metal chelates and proteins from the classes of protein A, protein L, protein A/G and protein G, is suitable.

Preferably, the agent is an antiserum antibody, which will bind specifically to more than one type of antibody, namely a secondary antibody against all antibodies of a given species. Since one antigen, having more than one epitope, evokes the production of more than one type of antibody, the analyte may comprise more than one type of antibody. This many-to-one ratio between the analyte and the agent plays in favor of the signal generation process by ensuring that an antigen-analyte immuno-binding event will be accompanied with another immuno-binding event between the analyte and the conjugate, thereby placing the second enzyme in proximity to the first enzyme and the electrode.

The first enzyme which is immobilized together with the antigen on the same surface is a member of an enzyme-channeling set, typically comprising two enzymes but may also comprise more. By definition, the first enzyme is capable of catalyzing the formation of a substrate of the second enzyme of a common enzymatic cascade.

The phrase “enzymatic cascade”, as used herein, relates to the phrase “enzyme-channeling” and describes a sequence of successive enzymatic reactions involving enzymes; each enzyme uses for a substrate the product of another enzyme in the cascade, the latter is therefore considered as “above” or “before” the former. Some enzymatic cascades are characterized by a series of amplifications of an initial stimulus or enzymatic reaction, such as, for example, in blood coagulation, wherein each enzyme activates the next until the final product, the fibrin clot, is formed.

Although cascades of two enzymes are described herein, the present invention encompasses similar systems that are based on cascades of three or more enzymes, which are selected suitable for effecting the generation of an electrochemical signal upon occurrence of the immunological event, namely the generation of a final product of the cascade which is an electrochemically detectable moiety, as defined herein. In cases where the immunological event is infrequent or otherwise rare, an amplification of the signal can be achieved by an enzymatic cascade which produces an exponentially increasing final product, thereby strengthening the electrochemical signal.

The immobilization of the antigen and the first enzyme is effected such that the two are immobilized proximally, namely located in sufficient proximity. The proximity of the antigen and the first enzyme, forces the strong coupling between the immunological event and the enzymatic cascade, exclusively near the electrode's surface. A more effective coupling of these events is effected by this proximity which creates a micro-environment wherein the concentrations of various solutes, such as the enzymes' substrates and products, are substantially higher near the electrode than in the bulk solution away from the electrode, and thus the enzymatic cascade reactions are not governed by diffusion-controlled process and rates across the entire electrochemical cell. This proximity-governed coupling enables the elimination of extensive washing steps, as discussed hereinbelow.

The phrase “immobilized proximally”, as used herein, refers to the immobilization of at least two entities, such as the antigen and the first enzyme, such that the physical distance between any one of the entities to the other is short in molecular terms, and in the order of magnitude of hundreds of angstroms or less to tenths of a micron. This proximal immobilization can be achieved by co-immobilizing these factors on a given surface at the same time and by the same reaction using a common reaction mixture for all entities, as demonstrated and successfully practiced in the Examples section that follows.

The purpose of coupling an enzymatic cascade to the immunoassay, is to produce an electrochemically detectable moiety in the system, thus, the second enzyme of the system presented herein generates an electrochemically detectable moiety upon binding of the conjugate to the antibody and binding of the antibody to the antigen on the electrode.

The phrase “electrochemically detectable moiety”, as used herein, refers to a substance which can accept or donate at least one electron during an electrochemical reaction, typically oxidation and/or reduction (redox), which occurs under controlled electrical conditions in an electrochemical cell. Each electrochemical event, namely an electron transfer to or from the electrochemically detectable moiety, contributes to the electrical current which the system can sense and record.

Therefore, the presence and/or amount of the electrochemically detectable moiety are detectable by the detecting unit of the system presented herein.

Since the enzymatic reaction of the second enzymes of the enzymatic cascade depends on the production of its substrate by the first enzyme, and since the second enzyme preferably produces an electrochemically detectible moiety, namely a moiety which can undergo a redox reaction on or near the electrode under a given mild potential, the selection of the co-dependent enzymes is initiated by the second enzyme.

The concept of enzymatic cascade and enzyme channeling is widely used for many applications. U.S. Pat. Nos. 5,516,644 and 6,406,876, which are incorporated by reference as if fully set forth herein, list several examples of suitable enzyme-sets which can be used in the context of the present invention.

In some cases one or more of the enzymes requires a secondary substrate for performing the catalysis. According to preferred embodiments of the present invention, the second enzyme requires the presence of a secondary substrate, such that it reacts with two substrates: one is provided by the first enzyme, and the other, referred to herein as a secondary substrate of the second enzyme, is separately added to the system presented herein, and participates in the enzyme channeling process.

According to preferred embodiments of the present invention, the first enzyme is a hydrogen peroxide producing enzyme.

The phrase “hydrogen peroxide producing enzyme” describes an enzyme which catalyzes a reaction that uses dissolved oxygen as a hydrogen acceptor or an electron donor to reduce another molecule (the oxidant, also called the electron acceptor) and during this redox reaction produces hydrogen peroxide as a by product.

Exemplary hydrogen peroxide producing enzymes include, without limitation, glucose oxidase (GOX, EC 1.1.3.4), glucose oxyhydrase, corylophyline, penatin, glucose aerodehydrogenase, microcid, β-D-glucose oxidase, D-glucose oxidase, D-glucose-1-oxidase, β-D-glucose:quinone oxidoreductase, glucose oxyhydrase, deoxin-1, nucleoside oxidase, NAD(P)H oxidase, hexose oxidase, L-sorbose oxidase and pyranose oxidase.

Preferably, the first enzyme is glucose oxidase (GOX, EC 1.1.3.4).

According to preferred embodiments of the present invention, the second enzyme is a peroxidase.

The term “peroxidase” describes an enzyme which catalyzes the oxidation of a substance by using a peroxide-containing molecule, typically hydrogen peroxide, as a hydrogen donor or an electron acceptor.

Exemplary peroxidases include, without limitation, horseradish peroxidase (HRP, EC 1.11.1.7), Japanese radish peroxidase, myeloperoxidase, lactoperoxidase, verdoperoxidase, guaiacol peroxidase, thiocyanate peroxidase, eosinophil peroxidase, extensin peroxidase, heme peroxidase, MPO, oxyperoxidase, protoheme peroxidase, pyrocatechol peroxidase, scopoletin peroxidase, L-ascorbate peroxidase, catalase, TPNH peroxidase, NADP peroxidase, nicotinamide adenine dinucleotide phosphate peroxidase, TPN peroxidase, triphosphopyridine nucleotide peroxidase, NADPH2 peroxidase, NADH peroxidase, iodide peroxidase, cytochrome-c peroxidase, manganese peroxidase and fatty-acid peroxidase.

Preferably, the second enzyme is horseradish peroxidase (HRP, EC 1.11.1.7).

The main part of the working electrode comprises a conductive material. The material can be selected according to preferred used of the electrode and the preferred mode of protein immobilization thereto. Preferably, the working electrode is selected form the group consisting of a conductive metal electrode and a conductive carbon electrode.

In order to produce a low-cost and disposable system, the working electrode is preferably a conductive carbon electrode such as, for example, a graphite electrode, a carbon ink electrode and a screen printed electrode. More preferably, the systems presented herein are based on the screen printed electrode technique, using carbon ink which is printed on an insulating electrode plate, including the working electrode.

Screen-printing technology is particularly attractive for the production of disposable sensors, such as used in the system presented herein. The “memory effect” between one sample to another is avoided by sidposal of a used electrode, and, the phenomenon referred to as “electrode fouling”, which is one of the main drawbacks of the electrochemical sensors, is overcome. Furthermore, these disposable sensors are characterized by high reproducibility and require no calibration.

Screen-printed electrodes are particularly useful in high-throughput screening (HTS) and ultra-high throughput screening (UHTS) technology. Their small size and low cost permit HTS/UHTS of large numbers of electrochemical assays to be conducted simultaneously, at minute volumes of microbiological and/or biochemical samples, using disposable, screen-printed electrochemical microarrays.

Alternatively, the working electrode is a conductive metal electrode such as, for example, a gold electrode, a platinum electrode, a silver electrode, a copper electrode, a nickel electrode, a chromium electrode, and a palladium electrode.

A prerequisite of the present system is having the enzyme and the antigen immobilized on the electrode is such a way that they substantially retain their three-dimensional structure and thus substantially retain their biological activity as a catalyst and an epitope, respectively. The enzyme and antigen may be immobilized on the surface of the electrode either directly or via an immobilization layer.

Hence, according to preferred embodiments of the present invention, the working electrode comprises an immobilization layer applied thereon, and the enzyme and antigen are immobilized on the working electrode via the immobilization layer.

The term “applied”, as used herein, refers to the spatial relations of close proximity between the surface of the electrode and the immobilization layer, hence, the immobilization layer may be attached to the electrode by adsorption; practically coat or plate the electrode, or be laid on the surface of the electrode as a separate sheet; sheathing the electrode and leaving a very small distance of a few tenths of a millimeter therebetween.

According to preferred embodiments of the present invention, the immobilization layer comprises a polymer attached to the surface of the working electrode and a cross-linking agent attached to the polymer.

According to these embodiments, the polymer coats the electrode by adsorption, thereby modifying its surface by adding reactive chemical functional groups to the surface of the electrode. Such chemical functional groups may include, without limitation, amines groups, hydroxyl groups, carboxyl group, thiol groups, aldehyde groups, hydrazide groups, diol groups, acyl groups, alkoxy groups, thioalkoxy groups, C-amide groups, N-amide groups and the likes.

Exemplary polymers suitable for adsorption of an electrode include, without limitation, polyethyleneimine, chitosan, polyethylene oxide, polyvinylalcohol, polyvinyl acetate, polyacrylamide, poly(vinylpyrrolidone), poly(2-vinylpyridine), poly(4-vinylpyridine), poly(4-vinyl-N-butylpyridinium) bromide and poly(vinylbenzyltrimethyl)ammonium hydroxide. Preferably, the polymer is a polyethyleneimine.

As used herein, the term “amine” refers to an —NR′R″ group where R′ and R″ are each hydrogen, alkyl, alkenyl, cycloalkyl, aryl, heteroaryl (bonded through a ring carbon) or heteroalicyclic (bonded through a ring carbon) as defined hereinbelow.

The term “alkyl” as used herein, describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 5 carbon atoms.

The term “alkenyl” refers to an alkyl group which consists of at least two carbon atoms and at least one carbon-carbon double bond.

The term “cycloalkyl” describes an all-carbon monocyclic or fused ring (i.e., rings which share an adjacent pair of carbon atoms) group where one or more of the rings does not have a completely conjugated pi-electron system. The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system.

The term “aryl” describes an all-carbon monocyclic or fused-ring polycyclic (i.e., rings which share adjacent pairs of carbon atoms) groups having a completely conjugated pi-electron system. The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

As used herein, the term “hydroxy” refers to an —OH group.

As used herein, the term “thiol” or “thiohydroxy” refers to an —SH group.

As used herein, the term “carboxyl” refers to an —C(═O)OR′ group, where R′ is as defined herein.

As used herein, the term “aldehyde” refers to an —C(═O)—H group.

The term “hydrazide”, as used herein, refers to a —C(═O)—NR′—NR″R′″ group wherein R′, R″ and R′″ are each independently hydrogen, alkyl, cycloalkyl or aryl, as these terms are defined herein.

As used in the context of the present invention, the term “diol” refers to a vicinal diol which is a —CH(OH)—CH(OH) group.

As used herein, the terms “acyl” and “carbonyl” refer to a —C(═O)-alkyl group, as defined hereinabove.

The term “alkoxy” as used herein describes an —O-alkyl, an —O-cycloalkyl, as defined hereinabove.

As used herein, the term “thioalkoxy” describes both a —S-alkyl, and a —S-cycloalkyl, as defined hereinabove.

As used herein, the term “C-amide” refers to a —C(═O)—NR′R″ group, where R′ and R″ are as defined herein.

As used herein, the term “N-amide” refers to an —NR′C(═O)—R″ group, where R′ and R″ are as defined herein.

The cross-linking agent, according to the present embodiments, acts as a linker between the chemical functional groups and free functional groups on the first enzyme and the antigen, and, by forming a web of interconnected residues thereof further contributes to the immobilization of the enzyme and the antigen. Exemplary cross-linking agents suitable for immobilizing the enzyme and the antigen include, without limitation, glutaraldehyde, polyglutaraldehyde, bis(imido esters), bis(succinimidyl esters), diisocyanates, succinimidyl acetylthioacetate, hydrazine, succinimidyl 3-(2-pyridyldithio)propionate, 3-(2-pyridyldithio)propionyl and tris-(2-carboxyethyl)phosphine. Preferably, the cross-linking agent is glutaraldehyde and/or polyglutaraldehyde.

Without being bound to any particular theory, it is assumed that the amount of the substrate of the second enzyme generated by the immobilized first enzyme is accumulated in the polymer, which enables electron transfer to the second enzyme, provided that the conjugate is bound to the analyte and to the immobilized antigen. A substrate of the second enzyme leaving the polymer and passing to the solution is diluted by orders of magnitudes and thus the activity of the free conjugates is decreased substantially and is not detectable if not found in close proximity to the electrode, thus the enzyme channeling enables the elimination of extensive wash steps.

According to preferred embodiments, when a secondary substrate is required for the second enzyme, and the second enzyme is a peroxide, it is required that the secondary substrate is capable of undergoing a redox transformation, namely to accept and electron from the electrode under specific cell potential and donate that electron during the peroxidase catalysis. Hence, the secondary substrate is preferably selected from the group consisting of potassium iodide (KI), p-phenylene diamine dihydrochloride (PPD) and acetaminophen.

The use of acetaminophen, also known as paracetamol, as the secondary substrate is highly advantageous since it is a common OTC drug of known and safe pharmacokinetic profile. It is therefore a user-friendly, safe and non-toxic component in the described system.

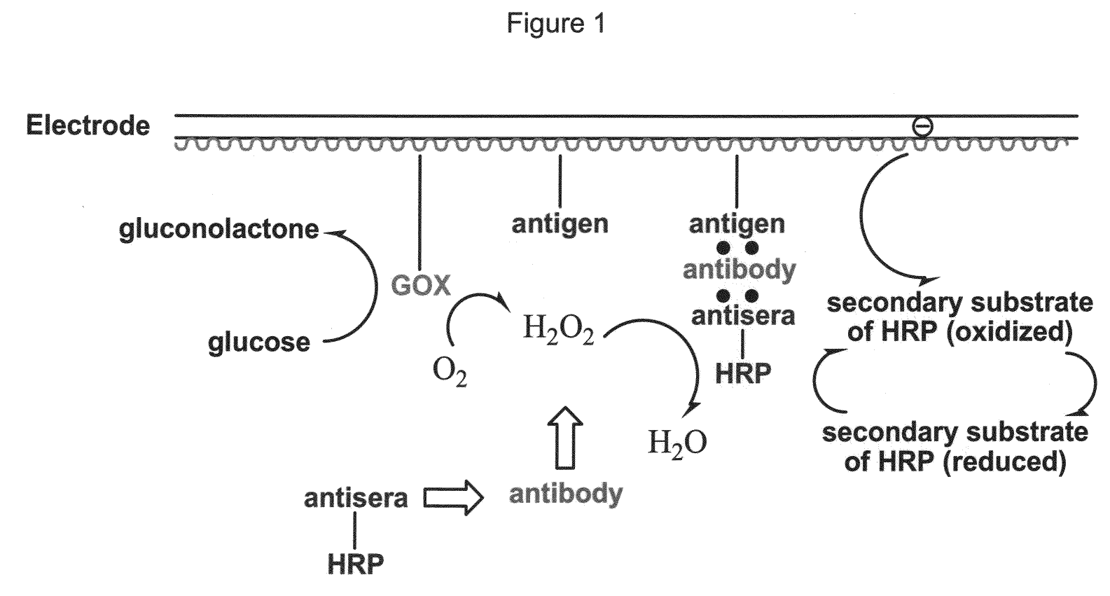

The gist of an exemplary system according to the present embodiments wherein the first enzyme and the antigen are attached to the working electrode by means of a polymeric/cross-linkable immobilization layer adsorbed thereto is illustrated in FIG. 1.

FIG. 1 depicts a system wherein glucose oxidase (GOX), serving as the first enzyme of the enzymatic cascade, and an antigen are attached to the immobilization layer (wavy line) which coats the working electrode. The system depicted in FIG. 1 further includes glucose as the substrate of the first enzyme which is converted to gluconolactone and hydrogen peroxide; the latter is the substrate of the second enzyme which is generated by the first enzyme. The system further includes a conjugate of an antisera antigen attached to horseradish-peroxidase (HRP), and an HRP substrate as a secondary substrate of the second enzyme. The electrochemically detectible moiety is produced and thereby a signal is recorded upon the combination of the following events:

the antibody (the analyte in question) binds to the antigen;

the antisera-antibody, conjugated to HRP, binds to the analyte;

hydrogen-peroxide which is concentrated near the electrode as a result of the enzymatic activity of the immobilized GOX and glucose, is reduced by HRP which also oxidizes the HRP secondary substrate; and

an electron transfer event is generated and recorded by the system.

All these events occur in proximity to the electrode, thus eliminating the need for wash steps and allowing a separation free immunoassay.

Alternatively, the immobilization layer comprises a microporous membrane, acting as a sheath which is laid on the surface of the electrode, and the antigen and the first enzyme are attached to this microporous membrane. The membrane desirably contains chemical functional groups which can interact with suitable free functional groups on the first enzyme and the antigen, and be permeable at least to the electrochemically detectable moiety, but can also be permeable to all the solutes in the electrochemical cell. In general, the membrane serves as a trap for the small molecules which are involved in the enzymatic cascade, such as the hydrogen peroxide, the secondary substrate and the electrochemically detectable moiety; hence it affects a local increase in the concentration of these compounds in the proximity of the working electrode by lowering their diffusion rate away from it. The proximity of electrochemically detectable moiety is a crucial prerequisite for the sensitivity and function of the system presented herein.

Suitable membranes, according to preferred embodiments, can be nitrocellulose-based membranes, and several commercially available membranes such as Immunodyne® ABC and Predator™ protein immobilization membranes.

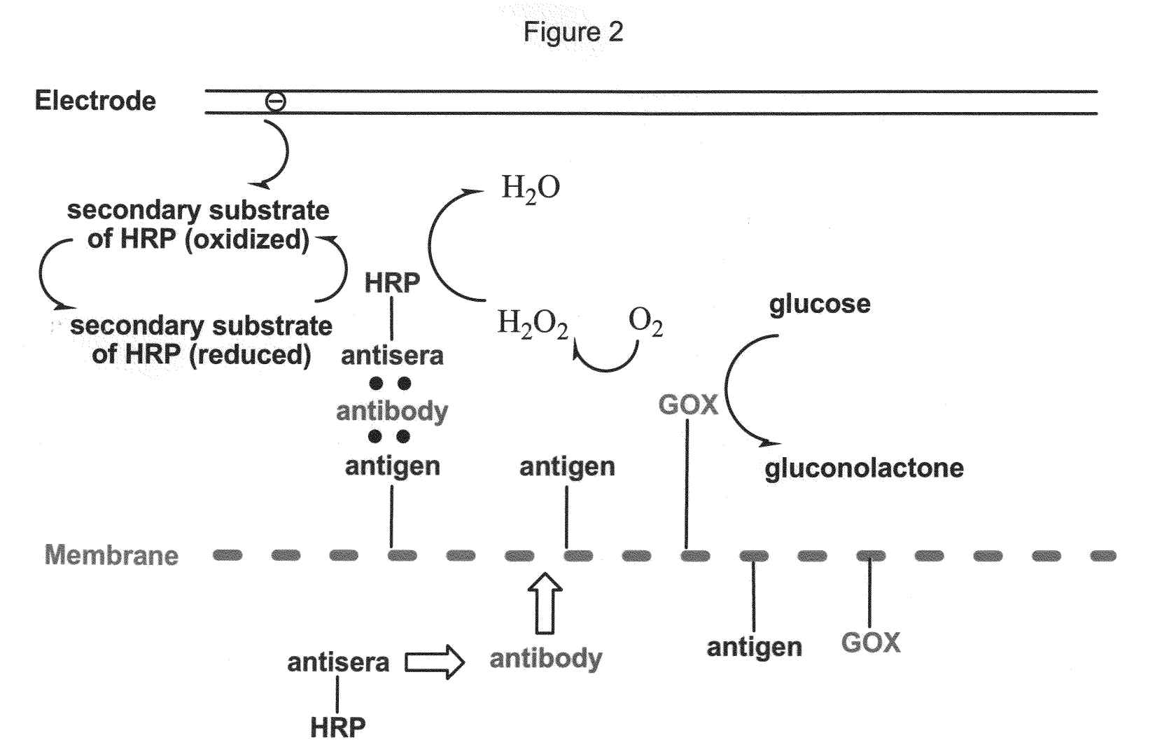

The gist of the system according to the present embodiments, wherein the first enzyme and the antigen are attached to a membrane which is laid on the working electrode is illustrated in FIG. 2.

FIG. 2 depicts a system wherein glucose oxidase (GOX), serving as the first enzyme of the enzymatic cascade, and an antigen are attached to a membrane, marked by a heavy dashed line, which is laid on the working electrode. The system depicted in FIG. 2 further includes glucose as the substrate of the first enzyme which is converted to gluconolactone and hydrogen peroxide; the latter is the substrate of the second enzyme which is generated by the first enzyme. The system further includes a conjugate of an antisera antigen attached to horseradish-peroxidase (HRP), and an HRP substrate as a secondary substrate of the second enzyme. The GOX and the antigen can be attached to both sides of the membrane, which places the latter in close proximity to the working electrode. Hydrogen peroxide is generated by GOX near the electrode and as in the case wherein the immobilizing layer coats the electrode, all the events occur in close proximity to the electrode, enabling a separation free immunoassay to be detected as presented herein.

Other protein immobilization techniques employing polymers and cross-linking agents are widely used and well established in the art can be used to immobilize the antigen and the first enzyme to the immobilization layer. These include, for example, techniques such as those taught in U.S. Pat. Nos. 3,933,589 4,272,617, 4,760,024, 5,071,909, 5,144,008, 5,258,502 and 5,279,948, which are all incorporated by reference as if fully set forth herein.

According to another aspect of the present invention there is provided a kit for detecting an antibody in a liquid sample, which includes a working electrode having immobilized thereon an antigen and a first enzyme of an enzymatic cascade as presented herein.

Depending on the antibody in question, namely the analyte, the conjugate, as presented herein, can be supplied as a part of the kit, or be supplied separately, or be provided as a commercially available reagent.

Similarly the substrate of the first enzyme and/or the secondary substrate of the second enzyme, as discussed herein, can be supplied as parts of the kit, or be supplied separately, or be provided as commercially available reagents.

The kit may be adapted to fit many commercially available electrochemical cells and systems, such that only the working electrode is provided in the kit, including or excluding the abovementioned reagents. Alternatively, the kit, according to preferred embodiments, may further contain a reference electrode, a counter electrode, an electrolytic solution and a current detecting unit. Further alternatively, the kit may contain all the abovementioned components, namely a comprehensive electrodes set (working-, counter- and reference-electrode), an electrolytic solution, a current detecting unit and all the reagents required for the analysis, namely the enzymes' substrates and the conjugate.

According to another aspect of the present invention, there is provided a working electrode for detecting an antibody in a liquid sample, the electrode includes a body and a surface having immobilized proximally thereon an antigen and a first enzyme of an enzymatic cascade, wherein the antigen is capable of specifically binding to the antibody, and the first enzyme is capable of catalyzing the formation of a substrate of a second enzyme in this enzymatic cascade, and wherein this second enzyme is capable of generating an electrochemically detectable moiety upon binding of a conjugate to the antibody and binding of the antibody to the antigen, whereby the conjugate comprises an agent capable of specifically binding to the antibody and the second enzyme of this enzymatic cascade being conjugated to this agent

Preferably, the surface of the electrode comprises an immobilization layer applied thereon, essentially as described hereinabove, wherein the antigen and the first enzyme of an enzymatic cascade are immobilized on the conductive element via the immobilization layer.

According to preferred embodiments, the conductive element comprises graphite, carbon ink, gold, platinum, silver, copper, nickel, chromium, and palladium, and more preferably, the conductive element comprises graphite and carbon ink.

As discussed hereinabove, the system and electrode presented herein are designed for detecting an antibody (the analyte) in a liquid sample, using a simple and reliable method. The system presented herein was successfully practiced to this end, as demonstrated in the Example section that follows.

Hence, according to another aspect of the present invention, there is provided a method of detecting an antibody in a liquid sample. The method, according to this aspect of the present invention is effected by:

contacting the liquid sample with a system, essentially as described hereinabove, which comprises:

an electrochemical cell which comprises:

a reference electrode, a counter electrode, a current detecting unit, an electrolytic solution and a working electrode having immobilized proximally thereon an antigen and a first enzyme of an enzymatic cascade, essentially as described hereinabove;

a substrate of the first enzyme; and

a conjugate which comprises an agent capable of specifically binding to the antibody and a second enzyme conjugated to the agent, essentially as described hereinabove.

According to this aspect, and as described in details hereinabove, the antigen is capable of specifically binding to the antibody, and the first enzyme is capable of catalyzing the formation of a substrate of the second enzyme, and further the second enzyme generates an electrochemically detectable moiety upon binding of the conjugate to the antibody and binding of the antibody to the antigen.

According to this aspect of the present invention, the presence and/or amount of the electrochemically detectable moiety is detectable by the detecting unit by routine and well established procedures.

An exemplary such procedure is effected by:

applying a pre-selected potential between the working electrode and the counter electrode, preferably subsequent to activating a power source which serves as an electron source for the working electrode;

recording a current formed between the working electrode and the counter electrode; and

determining the presence and/or amount of the electrochemically detectable moiety, thereby detecting the antibody (the analyte) in the liquid sample.

As discussed hereinabove, the system may further comprise a secondary substrate of the second enzyme.

According to embodiments of the present invention, the immunoassay can be performed by either adding all the components of the system at once to the electrochemical cell, referred to herein as a one-step mode, or by adding the components sequentially, in a specific order.

Therefore, according to preferred embodiments of the present invention, contacting the reaction mixture with the system comprises:

adding the liquid sample and the conjugate to the electrochemical cell, and subsequently adding to the cell the substrate of the first enzyme, to thereby initiate said enzymatic cascade.

Preferably, adding the liquid sample and adding the conjugate to the electrochemical cell is performed concomitantly.

Alternatively, adding the liquid sample and adding the conjugate to the electrochemical cell is performed sequentially.

In both cases, it is preferred to allow the conjugate to incubate with the liquid sample containing the analyte antibody so as to allow these two components to bind to one another, and to allow the analyte to bind to the antigen before the substrate of the first enzyme and optionally the secondary substrate of the second enzyme, is/are introduced into the cell. The sequential addition of the reaction components may be needed in some cases where the analyte is present in a relatively low concentration, or when the antigen is recognized by a small number of types of antibodies.

The incubation time will also allow the members of the enzymatic cascade to accumulate near the electrode before one or more of the substrates is processed by the enzymes, hence, according to preferred embodiments, contacting the reaction mixture with the system comprises:

adding the liquid sample and the conjugate to the electrochemical cell;

subsequently adding the secondary substrate to the electrochemical cell; and

subsequently adding to the cell the substrate of the first enzyme.

In cases where the second enzyme requires the presence of a secondary substrate, the adding the liquid sample and the conjugate to the electrochemical cell may be performed either concomitantly with the addition of the secondary substrate or by adding the liquid sample, the conjugate and the secondary substrate sequentially.

Alternatively, since the enzymatic cascade cannot commence until the first enzyme produced the substrate of the second enzyme, adding the liquid sample, the conjugate and the secondary substrate to the electrochemical cell may be performed concomitantly, and adding the substrate of the first enzyme is performed subsequent to adding the liquid sample and the conjugate.

Several of these various addition sequences are demonstrated in the Example section that follows.

As discussed hereinabove, the enzymatic cascade offered by hydrogen peroxide producing enzymes, primarily from the oxidase family, together with enzymes of the peroxidase family, constitutes a preferred enzyme channeling set. Yet, the need of a secondary substrate for the second enzyme which, upon commencement of the enzymatic cascade, is converted to an electrochemically detectible moiety; a crucial component of the entire system and method, requires the use of substances which are oftentimes unstable and toxic, as is often the case with many redox-prone substances.

While conceiving the present invention, the present inventors hypothesized the use of a commonly used and highly safe secondary substrate. While reducing the present invention to practice, the system described above successfully employed acetaminophen as a secondary substrate of a peroxidase second enzyme, as demonstrated in the Examples section that follows.

These findings suggest that systems similar the systems described hereinabove, which are based on other binding pairs, electrochemical cells in general and biosensors in particular, can be beneficially operated by using acetaminophen as a secondary substrate of a peroxidase or other enzymatic mechanisms which require the use of a safe and stable electron acceptor/donor molecule.

Hence, according to an additional aspect of the present invention, there is provided a system for detecting a first member of a binding pair in a liquid sample, the system comprising components essentially as described hereinabove, except for the first enzyme of the enzymatic cascade being a hydrogen peroxide-producing enzyme, the second enzyme of the enzymatic cascade being a peroxidase, and the secondary substrate is acetaminophen.

This system, suitable for detecting any member of a binding pair using the same concept of proximal enzymatic cascade effected by immobilizing one member of the binding pair in proximity to the first enzyme of the enzymatic cascade, and binding of the other member of the binding pair to a conjugate which includes an agent capable of specifically binding to the first member of the binding pair, and the second enzyme attached thereto.

As in the system described hereinabove, the second enzyme generates a detectable form of acetaminophen upon binding of the conjugate to the first member of the binding pair and binding of the first member to the second member of the binding pair. In turn, the presence and/or amount of this detectable form of acetaminophen are recorded by the detecting unit.

This more general system allows either one of the binding pair to be the analyte while its counterpart is immobilized on the working electrode.

Binding pairs which are suitable for use within this context of the present invention include, for example, a receptor—ligand binding pair, an enzyme—inhibitor binding pair, an enzyme—substrate binding pair, polynucleotide sequence—complimentary polynucleotide sequence binding pair and an antigen—antibody binding pair.

According to another aspect of the present invention, there is provided a method of detecting a first member of a binding pair in a liquid sample, essentially as described hereinabove, by contacting the liquid sample with a system which comprises the acetaminophen-based detection system described hereinabove, to thereby detect the presence and/or amount of the detectable form of acetaminophen, and thereby detecting the any one member of a binding pair in a liquid sample.

As in the case of the method described hereinabove, the components of the reaction mixture can be added in sequence or concomitantly.

For example, adding the liquid sample and the conjugate to the electrochemical cell, adding the acetaminophen to the electrochemical cell, and subsequently adding the substrate of the first enzyme to the cell.

Alternatively, adding the liquid sample and the conjugate to the electrochemical cell concomitantly, or adding the liquid sample, the conjugate and the acetaminophen to the electrochemical cell concomitantly; or adding the liquid sample, the conjugate and the acetaminophen to the electrochemical cell sequentially; or adding the liquid sample, the conjugate and the acetaminophen to the electrochemical cell concomitantly, and adding the substrate of the first enzyme subsequent to adding the liquid sample and the conjugate.

All the aforementioned methods for detecting an antibody or a member of a binding pair, according to the present invention, can be based on the unique design of the system and the electrode presented herein, therefore the detection procedure can be performed in a separation free mode wherein the contacting is effected without washing the cell.

The separation free mode relies on the attenuation and minimization of non-specific interactions and substrate consumption away from the electrode. This requirement can be provided by using low concentrations of the conjugate with respect to the immobilized antigen or the immobilized member of the binding pair. Thus, according to preferred embodiments, when using the systems and methods presented herein in a separation free mode, the molar ratio of the conjugate and the antigen or member of a binding pair ranges from about 1:100 to about 1:10,000, preferably the molar ratio ranges from about 1:100 to about 1:5,000, and most preferably the molar ratio is about 1:1000.

Alternatively, these methods can be performed in such a mode wherein the contacting further comprises washing the electrochemical cell upon adding the liquid sample and/or upon adding the conjugate.

According to preferred embodiments, the molar ratio between the antigen or the immobilized member of the binding pair and the first enzyme ranges from about 1:5 to about 5:1. More preferably, the molar ratio between the antigen and the first enzyme ranges from about 1:2 to about 2:1, and most preferably, the molar ratio between the antigen and the first enzyme is about 1:1.

According to preferred embodiments of the present invention, the detection of the analyte, either an antibody or another analyte which is a member of a binding pair, is performed qualitatively.

More preferably, the detection of the analyte, either an antibody or another analyte which is a member of a binding pair, is performed quantitatively. Typically, quantitative determination of the analyte is based on the use of standard solutions of the analyte or another substance which provokes a similar binding event between the antigen or the immobilized member of the binding pair and the corresponding agent forming a part of the conjugate with the second enzyme of the enzymatic cascade.

In general, the systems, kits, methods and electrodes presented herein are highly suitable for on-the-spot determination, either qualitatively or quantitatively, of an immune response in a subject, using a liquid sample extracted therefrom, such as a blood or serum sample.

Preferably, the immune response is selected from the group consisting of an immune response to a pathogenic microorganism including fragments thereof such as a protein, a peptide, a membrane and other viral or bacterial components, an immune response to a toxin, an immune response to a drug, an immune response to a foreign particle, an immune response to an organ transplant and an immune response to an implant.

As demonstrated in the Examples section that follows, the system and method presented herein were used to quantitatively determine the level of an immune response of a subject to a pathogenic microorganism. More specifically, these experiments were conducted so as to show that the enzyme-channeling coupled immunoassay concept can be used to determine the vaccination level titer in dogs. Therefore, the immunoassay was conducted for serum samples extracted from dogs in order to determine the titer level of antibodies which were produced against a canine pathogen, and more specifically, the canine pathogen was a canine distemper virus, constituting a preferred embodiment of the present invention.

Alternatively, the systems, kits, methods and electrodes presented herein can be used to determine the level of antibodies production in an in vitro and/or an artificial environment, such as hybridoma conditioned media.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Experimental Methods

Graphite electrodes were prepared by extracting the graphite from common pencils obtained from “Dyonon” Tel-Aviv University student shop.

Screen printed electrodes (SPE) were obtained from Gwent Electronics, England.

Glucose oxidase (GOX), horseradish peroxidase (HRP), bovine serum albumin (BSA), glutaraldehyde (GA), polyethyleneimine (PEI), biotin-HRP and p-phenylene diamine dihydrochloride (PPD) were obtained from Sigma, Israel.

Dog immunoglobulin G (IgG) and anti-dog-IgG-HRP (α-dog-IgG-HRP) was obtained from Jackson ImmunoResearch laboratories Inc. (West Grove, Pa., USA).

Canine Distemper antigen virus (CDV) and dog sera were obtained from Biogal, Galed Lab., Kibbutz Galed, Israel.

Immunodyne® ABC membrane was obtained from Pall Corporation (East Hills, N.Y., USA).

Predator™ membrane was obtained from Pall Gelman Sciences Inc. (Ann Arbor, Mich., USA).

Nitrocellulose membrane was obtained from Schleicher & Schuell BioScience, Inc. (Keene, N.H., USA).

### Preparation of Antigen-Loaded Graphite Electrodes

The graphite electrodes were polished on a paper sheet, washed in double distilled water (DDW), sonicated for 10 minutes in ethanol, then washed again in DDW and left to dry at room temperature.

Amine groups, free for binding, were added to the cleaned surface of the working electrodes by treatment with a methanol solution having 0.05% polyethyleneimine (PEI) for 1 hour at room temperature. Thereafter the electrodes were washed with DDW and left to dry at room temperature.

Aldehyde groups were added to the working electrodes by covalently attaching glutaraldehyde (GA) to the free amine groups of the PEI. The PEI-treated electrodes were placed in 600 μl tubes containing 200 μl of an aqueous solution containing 0.25% glutaraldehyde in 0.1M phosphate buffer (pH 7.5) for 1 hour at room temperature. Subsequently, the electrodes were washed in 0.1M phosphate buffer (pH 7.5) and left to dry at room temperature.

Antigens and antibodies, jointly referred to herein as the epitope-containing agent (ECA), namely canine distemper antigen virus (CDV), anti-dog-IgG (α-dog-IgG) or dog-IgG, were covalently attached to the working electrodes via the free aldehyde groups attached thereto by incubating the PEI/GA treated electrodes in an aqueous solution (20 μl) containing 0.5 mg/ml of the ECA and 0.5 mg/ml glucose oxidase (GOX). For use in control experiments, the PEI/GA treated electrodes were incubated in an aqueous solution containing 0.5 mg/ml BSA and 0.5 mg/ml GOX for 1 hour at room temperature.

The electrodes were washed with 0.1 M phosphate buffer (pH 7.5), and incubated in an aqueous solution containing 0.1 M phosphate buffer (pH 7.5) and 0.1 M glycine for 1 hour at room temperature, so as to block free amine groups which were left on the surface of the electrodes. Thereafter the electrodes were washed with 0.1 M phosphate buffer (pH 7.5) and further blocked for non specific binding with 1% BSA in 0.1 M phosphate buffer (pH 7.5) for 1 hour at room temperature.

The ECA/GOX- or BSA/GOX-loaded graphite electrodes were washed and kept in 0.1 M phosphate buffer (pH 7.5) at 4° C.

### Preparation of Screen Printed Electrodes (SPE)

Screen printed electrodes (SPE) having an antigen attached thereto were prepared essentially according to the above procedure with some modifications, as follows.

Three μL of 0.05% polyethyleneimine diluted in DDW, were positioned onto the working electrode and left to dry for 1 hour at room temperature. After washing the surface of the SPE with DDW, an aliquot of 3 μl of an aqueous solution having 0.25% glutaraldehyde in 0.1 M phosphate buffer (pH 7.5) was deposited onto the PEI-treated SPE, and the electrodes were left to dry for an hour at room temperature. Thereafter the electrodes were washed with 0.1 M phosphate buffer (pH 7.5) and the free amine groups were blocked with 0.1 M glycine in 0.1 M phosphate buffer (pH 7.5) for an hour at room temperature.

After washing the glycine-treated SPE, 3 μl of an aqueous solution containing both GOX (0.5 mg/ml final concentration) and an ECA, namely dog-IgG (0.5 mg/ml) or canine distemper virus antigen (CDV, original stock solution was diluted 1:20 to reach a 0.05 mg/ml final concentration), or avidin (AV, 0.5 mg/ml), were deposited thereon for incubation of 1 hour at room temperature. SPE samples treated with BSA (0.5 mg/ml) instead of an ECA or avidin were prepared for use in control experiments.

Thereafter the ECA- or avidin- or BSA-loaded SPE were further washed and blocked for remaining amine reactive groups with 0.1M glycine in 0.1 M phosphate buffer (pH 7.5) for 1 hour at room temperature, further blocked with 1% BSA, gelatin or skim milk in 0.1 M phosphate buffer (pH 7.5) for 1 hour at room temperature.

The ECA- or avidin- or BSA-loaded screen printed electrodes were washed and kept in 0.1 M phosphate buffer (pH 7.5) at 4° C.

### Membranes Preparation

Immunodyne® ABC, Predator™ or nitrocellulose membrane were cut to pieces of 0.9 cm×0.9 cm or 0.9 cm×0.25 cm and placed in a small vessel containing a solution of 0.1 M phosphate buffer (pH 7.5). Thereafter 4 μl of each of the following aqueous solutions containing 0.1 M phosphate buffer (pH 7.5) were deposited drop-wise on top and center of a membrane piece, to form the following types:

| | |
|---|---|
| Type-A: | 0.5 mg/ml GOX + 0.5 mg/ml dog-IgG; |
| Type-B: | 0.5 mg/ml GOX + 0.05 mg/ml CDV; |
| Type-C: | 0.5 mg/ml GOX + 0.5 mg/ml avidin (AV); |
| Type-D: | 0.5 mg/ml GOX + 0.5 mg/ml BSA for control experiments. |

All membrane types were thereafter dried for 1 hour at room temperature, washed with 0.1 M phosphate buffer (pH 7.5) and blocked with 0.1 M phosphate buffer pH 7.5 containing 1% BSA, gelatin or skim milk for 1 hour at room temperature. All dilutions used 0.1 M phosphate buffer (pH 7.5).

After washing with 0.1 M phosphate buffer (pH 7.5), samples of the membranes of Type-A and Type-B, loaded with GOX/dog-IgG and loaded GOX/CDV respectively, were incubated with 1:1000 diluted conjugate of α-dog-IgG and horseradish peroxidase (α-dog-IgG-HRP) and washed again with buffer.

Samples of membranes of Type-B and Type-D, loaded with GOX/CDV and loaded GOX/BSA respectively, were incubated with positive and negative dog sera (diluted 1:100). After the incubation with dog sera the membranes were washed and blocked with 0.1 M phosphate buffer (pH 7.5) containing 1% BSA, gelatin or skim milk for 1 hour at room temperature.

### Avidin-Biotin Model Platform

In order to construct a general measurement platform for a wide range of antigens and antibodies, a simplified model platform of known and anticipated characteristics was constructed based on the avidin-biotin affinity pair, which replaces the antigen-antibody affinity pair. The electrodes were prepared essentially as described hereinabove by co-immobilization of GOX and avidin onto the PEI modified graphite electrodes.

Biotinilation of Canine Distemper Virus Antigen (CDV)

Distemper virus was diluted 1:10 in 0.1 M phosphate buffer (pH 7.5) to a total volume of 1 ml. The antigen solution was dialyzed through a dialysis membrane (50,000 Dalton cut-off) against the same buffer at 4° C. Protein concentration was determined by optical density at 280 nm according to extinction coefficient of 1 OD correlation to 1 mg/ml.

The dialyzed CDV antigen was further diluted 1:10 in 0.1 M sodium borate buffer (pH 8.8) to a final concentration of 1.3 mg/ml.

N-Hydroxysuccinimide biotin was dissolved in dimethyl sulfoxide (DMSO) to a concentration of 10 mg/ml.

5 μL of the N-hydroxysuccinimide biotin solution was added to 1 ml of CDV antigen solution with biotin final concentration of 50 μg/ml. The mixture left to react for 4 hours at room temperature. The reaction was quenched by the addition of 4 μL of 1 M NH₄Cl and incubation for 10 minutes.

The biotinilated CDV antigen was dialyzed extensively against phosphate buffer and stored at −20° C.

### Electrochemical Measurement Apparatus

Graphite Electrodes

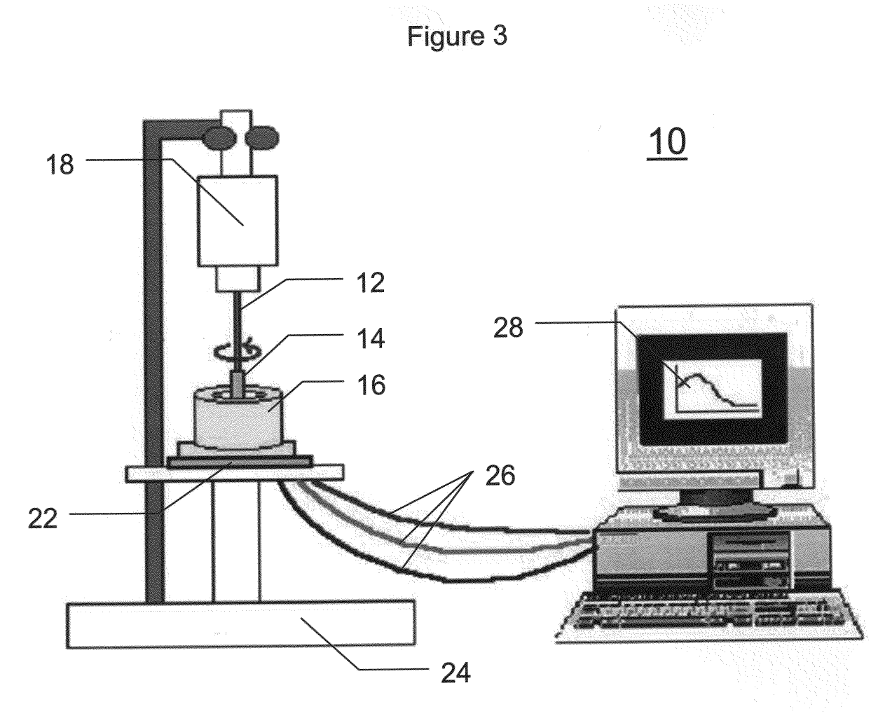

The electrochemical cell in which graphite electrodes were used is schematically illustrated in FIG. 3. As can be seen in FIG. 3, the electrochemical cell 10 was fitted with a graphite electrode 12 which was used as a working electrode. The graphite electrode 12 was connected to the rotating device 18 and shafted through a silicon coat 14. The electrochemical cell 10 further comprised a measuring Teflon cylinder 16, two screen-printed electrodes, namely a carbon counter electrode and an Ag/AgCl reference electrode (provided by Prof. C. McNeil, Newcastle, England) placed on an electrode plate 22 underneath the measuring Teflon cylinder 16, and a potentiostat 24. The electrodes were connected by electrode lines 28 to the central control and signal processing unit 28.

Screen Printed Electrodes (SPEs)

The goal of experiments using SPEs was to show feasibility of the system with a disposable triad of SPEs containing all three electrodes in one electrical circuit printed on one electrode plate. Disposable electrodes are advantageous for development of non-invasive sensors, especially with “on the spot” monitoring immunosensors. These kinds of electrodes are easy to handle and eliminate the need for another electrode such as a graphite electrode, which requires additional electrical wiring and accessories. The modified PEI polymer was deposited on the carbon ink printed working electrode by the same procedures described for the graphite working electrode.

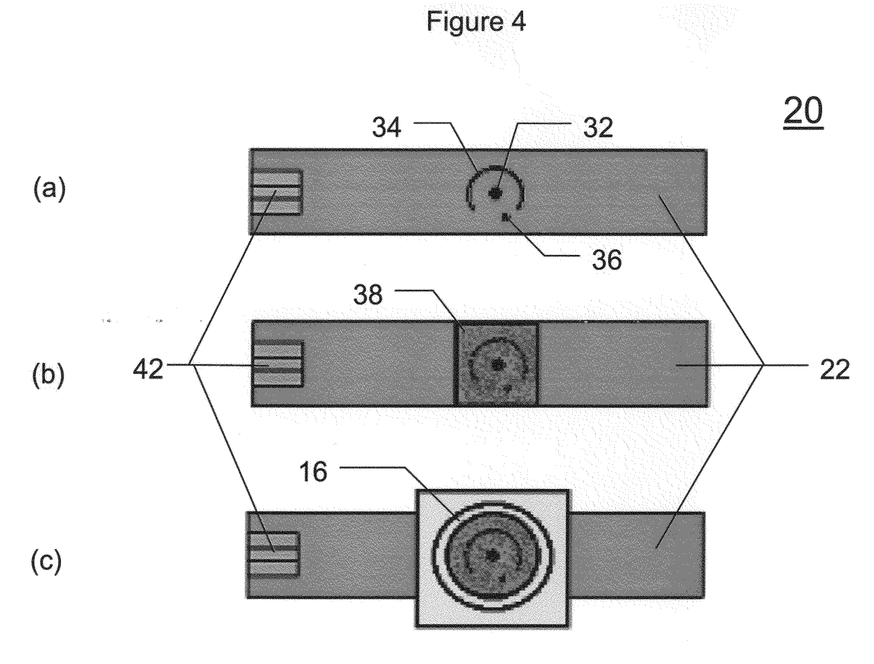

The SPE electrodes system 20, shown in FIG. 4a, was used in an electrochemical cell as described for the graphite electrode hereinabove, and consisted of three screen printed electrodes. As can be seen in FIG. 4a, a carbon ink working electrode 32, a carbon ink counter electrode 34 and an Ag/AgCl reference electrode 36 (Gwent, England), were printed on an electrode plate 22 having electrical connectors 42 which connected the electrodes system 20 to the central control and signal processing unit via the electrode lines.

Membranes and SPE Electrodes

A membrane and SPE electrodes system, shown in FIGS. 4b and 4c, was used in an electrochemical cell as described for the graphite electrode hereinabove, and consisted of an electrodes system 20 as described hereinabove, and a membrane 38, previously treated with serum or relevant antibodies, which was placed on top of the three screen printed electrodes. A measuring Teflon cylinder 16 was placed over the membrane and the electrodes so as to form the measuring container.

### Preparation of Experimental Solutions for Electrochemical Measurements

Graphite Electrodes

Experimental “no-wash; one-step” measurements using the modified graphite electrodes were conducted by introducing α-dog-IgG-HRP conjugates or biotin-HRP, both diluted 1:1000 with the working buffer containing 0.1 M phosphate buffer (pH 5.8) 0.1 M KCl, 1% BSA, 0.01% Tween-20, 1 mM glucose and 2 mM of an HRP substrate such as potassium iodide (KI), p-phenylene diamine dihydrochloride (PPD) or N-(4-hydroxy-phenyl)-acetamide (also known as paracetamol or acetaminophen).

Screen Printed Electrodes

Experimental “no-wash; one-step” measurements using the GOX/AV modified screen printed electrodes were conducted by introducing positive or negative dog sera diluted 1:100 with the working buffer containing 0.1 M phosphate buffer (pH 5.8) 0.1 M KCl, 1% BSA, 0.01% Tween-20, 1 mM glucose and 2 mM of an HRP substrate as presented hereinabove.

Membranes

The preferred electrodes for disposable measurement devices are screen printed electrodes. Since a disposable sensor is required to measure blood samples without the addition of a buffer, the laminar flow immunosensor was designed in analogy to a typical commercial pregnancy kit, thus laminar flow membrane, such as Predator™, was chosen. This immunosensor is based on a laminar flow membrane that passes the analytes through the working electrode surface, thus forming a peak shaped signal.

Experimental “wash” measurements were conducted by placing the ECA-, AV- or BSA-loaded and washed membranes onto the screen printed electrodes in an electrochemical cell and introducing α-dog-IgG-HRP conjugates diluted 1:1000 with the working buffer containing 0.1 M phosphate buffer (pH 5.8), 0.1 M KCl, 1% BSA, 0.01% Tween-20, 1 mM glucose and 2 mM of an HRP substrate compound as presented hereinabove.

Experimental “no-wash, one-step” measurements using the membranes prepared as described hereinabove were conducted by placing a membrane piece onto the SPE in an electrochemical cell without washing, and introducing α-dog-IgG-HRP conjugates, diluted 1:1000 with the working buffer containing 0.1 M phosphate buffer (pH 5.8), 0.1 M KCl, 1% BSA, 0.01% Tween-20, 1 mM glucose and 2 mM of an HRP substrate compound as presented hereinabove. Introduction of α-dog-IgG-HRP conjugates was conducted in the absence or the presence of positive or negative dog sera.

### Experimental Procedures

Measurement Potentials

For measurements with p-aminophenylphosphate (pAPP, 1 mg/ml final concentration) and alkaline phosphatase conjugated α-dog-IgG (original stock diluted 1:1000), the working potential was 220 mV.

For measurements with KI working potential was 0 mV.

For measurements with PPD working potential was −50 mV.

For measurements with acetaminophen working potential was −100 mV.

Measurements with Wash Steps

The graphite or SPE electrodes were extensively washed in 0.1 M phosphate buffer pH 7.5 and thereafter were used for measurements in an electrochemical cell as described hereinabove. The total volume of the reaction solution was 300 μl or 970 μl which included 0.1 M phosphate buffer (pH 5.8), 0.1 M KCl and 0.01% Tween-20. The substrates, KI (3 μl, 1 mM final concentration), PPD (3 μl, 1 mM final concentration) or acetaminophen (6 μl, 1 mM final concentration), all accompanied by glucose (6 μl, 2 mM final concentration), were added a few seconds after commencing the recording of the electronic signals coming from the electrodes.

Separation-Free Measurements without Wash Steps

The graphite or SPE electrodes were used for “separation-free” measurements in an electrochemical cell as described hereinabove. The total volume of the reaction solution was 300 μl or 970 μl which included 0.1 M phosphate buffer (pH 5.8), 0.1 M KCl and 0.01% Tween-20, in the presence of the appropriate conjugate at 1:1000 final dilution. The substrates, KI (3 μl, 1 mM final concentration), PPD (3 μl, 1 mM final concentration) or acetaminophen (6 μl, 1 mM final concentration), all accompanied by glucose (6 μl, 2 mM), were added a few seconds after commencing the recording of the electronic signals coming from the electrodes.

One-Step, Separation-Free Measurements without Wash Steps

The graphite or SPE electrodes were used for “one-step, separation free” measurements in an electrochemical cell as described hereinabove. Prior to introduction of the conjugates, 150 μl of a solution containing 0.1 M phosphate buffer (pH 5.8), 0.1M KCl and 0.01% Tween-20 were placed in the electrochemical cell. A few seconds after commencing the recording of the signal from the electrodes, additional 150 μl of this solution, containing the appropriate conjugate (final dilution of 1:1000), acetaminophen (1 mM final concentration) and glucose (2 mM final concentration) were added to the electrochemical cell.

Laminar Flow Measurements

The laminar flow membrane was prepared as described hereinabove, and the membrane was placed on the surface of the screen-printed electrode on the side of the working electrode. An absorbent pad was placed on the other side of the electrode plate in order to drive a streamline flow of the measurement solutions. The measurement solution contained α-dog-IgG-HRP at various dilutions, dog serum (1:100 dilution) and substrates at the abovementioned concentrations. The measurement solution (100 μl) was applied drop-wise on one side of the membrane, which flowed nonturbulently through the membrane and the expected signal was recorded as a peak when the solution flowed through the electrode area and came in contact with the immobilized antigen or antibody. Similar experiments were conducted with immobilized antigens using PEI-treated SPEs.

Test Samples for Membrane-Based Immunoassays

The following test samples, containing analytes and control sample at various titers were used for determining the validity and accuracy of the Immunodyne® ABC membrane-based immunosensors presented herein, prepared with two relative ratios of the immobilized GOX to antigen, achieved by two dilution ratios with respect to the antigen's sample as supplied by the vendor, namely 1:10 dilution, reaching 0.1 mg/ml final concentration and denoted “1:10Ag”, and 1:1 (undiluted), reaching 1 mg/ml final concentration and denoted “1:1Ag”:

1. Dog serum positive for CDV measured using a membrane prepared with a 1:1 antigen dilution, denoted “positive serum770(1:1 Ag)”, wherein “serum770” refers to the vendor's sample serial number;

2. Dog serum positive for CDV measured using a membrane prepared with a 1:10 antigen dilution and denoted “positive serum770(1:10 Ag)”;

3. Dog serum positive for canine parvovirus disease (CPVD, one of the typical infectious diseases in dogs) at low levels, measured using a membrane prepared with a 1:1 antigen dilution and denoted “low level serum (1:1)CPDV”;

4. Dog serum positive for canine parvovirus disease (CPVD, one of the typical infectious diseases in dogs) at low levels, measured using a membrane prepared with a 1:10 antigen dilution and denoted “low level serum (1:10)CPDV”;

5. Dog serum negative for all diseases, measured using a membrane prepared with a 1:1 antigen dilution and denoted “negative serum#4(1:1Ag)”;

6. A duplicate experiment according to sample No. 5.

7. Dog serum negative for all diseases, measured using a membrane prepared with a 1:10 antigen dilution and denoted “negative serum#4(1:10Ag)”;

8. A duplicate experiment according to sample No. 7.

9. A control sample containing no serum, measured using a membrane prepared with a 1:1 antigen dilution and denoted “no serum(1:1Ag)”;

10. A duplicate experiment according to sample No. 9.

11. A control sample containing no serum, measured using a membrane prepared with a 1:10 antigen dilution and denoted “no serum(1:10Ag)”; and

12. A duplicate experiment according to sample No. 11.

### Experimental Results

### Measurements Using Purified Reagents

Membrane-Based Immunoassays:

The distemper virus antigen or BSA was covalently immobilized onto Immunodyne® ABC membrane at different dilutions (1:1 and 1:10) as described hereinabove. After blocking and washing, the membranes were incubated with dog serum with different titer levels followed by incubation with α-dog-IgG-HRP. After extensive wash steps the membranes were laid onto the SPEs as described hereinabove and the signal generated by the enzymatic reaction was recorded.

The results of these experiments were compared to the results obtained with a commercial Biogal immunological system kit which were used according to the specification provided with the kit. The experiments using the commercial kit were conducted with extensive wash steps between each stage in analogy to typical ELISA procedures, and without employing the bi-enzyme-channeling signal generation.

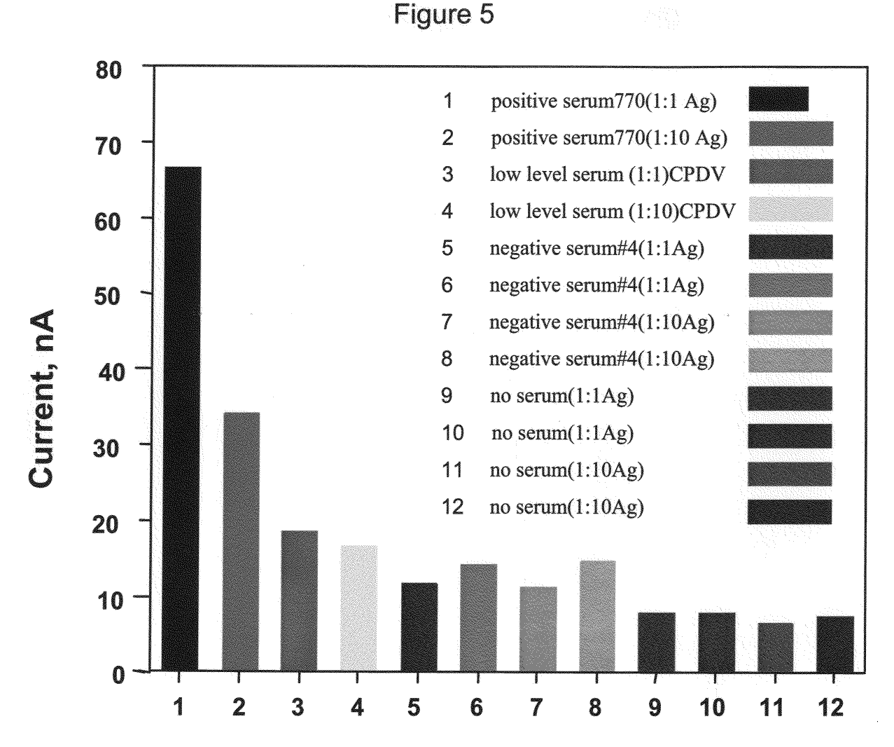

In general, the signal obtained by the present immunosensors exhibited high correlation to the immobilized antigen density of the membrane and with the serum titer level for the measured samples. Moreover, the results obtained by the present immunosenors agreed with the results obtained by the commercial Biogal immunological system kit, and the resulting electric signals are presented in FIG. 5.

FIG. 5 presents a comparative bar diagram, showing the maximal signal recorded in various experiments, which are color-coded as follows:

1. “positive serum770(1:1 Ag)” in black;

2. “positive serum770(1:10 Ag)” in red;

3. “low level serum (1:1)CPDV” in light green;

4. “low level serum (1:10)CPDV” in yellow;

5. “negative serum#4(1:1Ag)” in blue;

6. “negative serum#4(1:1Ag)” in magenta;

7. “negative serum#4(1:10Ag)” in cyan;

8. “negative serum#4(1:10Ag)” in gray;

9. “no serum(1:1Ag)” in brown;

10. “no serum(1:1Ag)” in dark green;

11. “no serum(1:10Ag)” in olive; and

12. “no serum(1:10Ag)” in navy blue.

As can be seen in FIG. 5, the signals recorded using a membrane which was prepared with a 1:1 antigen dilution ratio, namely undiluted, were noticeably higher for samples of positive samples as compared to signals obtained with a membrane having a tenth of the immobilized antigen attached thereto. Accordingly, signals measured for sample without the presence of an analyte (negative sera or no sera) were exhibited an almost indistinguishable difference with respect to the antigen density.

These results clearly demonstrate the validity and capacity of the present immunosensors in quantitatively detective an immunological analyte in an unknown sample.

Successive Steps, No-Wash and Separation-Free Immunoassay Using Graphite Electrode and PPD as HRP Secondary Substrate:

The results of the following experiment demonstrated the concept and use of enzyme channeling for “no-wash” immunoassays, using graphite electrodes (pencil leads), artificial polymer PEI and HRP-substrate PPD as substrate.

A graphite working electrode was coated by absorption with PEI polymer modified with glutaraldehyde for co-immobilization of GOX and dog-IgG or BSA. The electrochemical cell, described hereinabove, comprised the graphite (pencil lead) working electrode, a carbon ink counter SPE and an Ag/AgCl reference SPE. A 300 μl measuring Teflon cylinder was assembled on the electrode plate and served as the reaction cell.

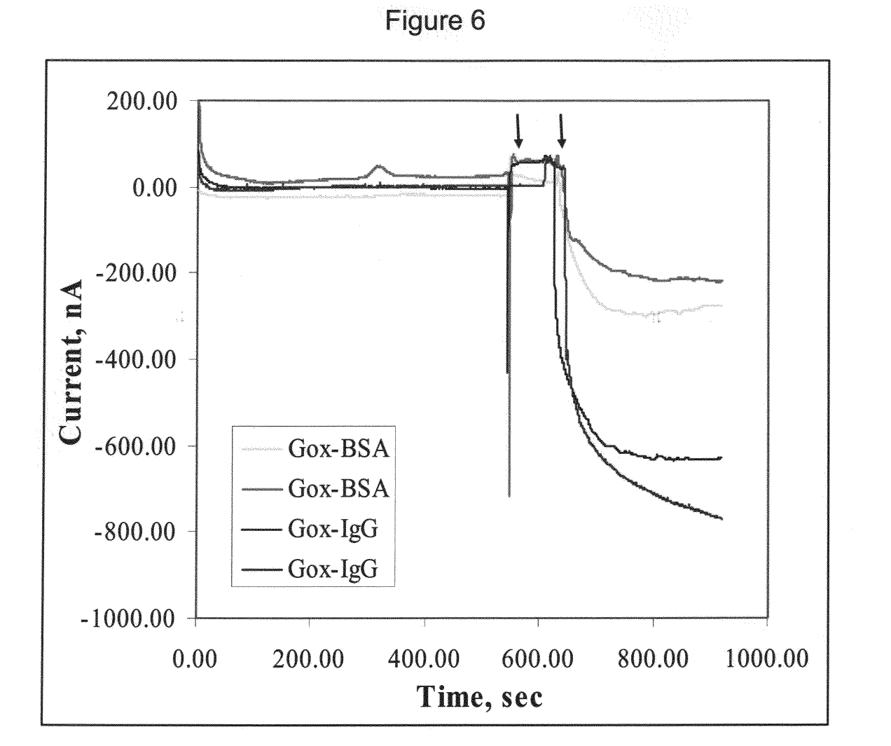

The assay was conducted without any wash steps, by successive additions of the substrates and the conjugate at 50 seconds intervals, namely glucose, PPD and α-dog-IgG-HRP (diluted 1:1000), to the electrochemical cell, and the results are presented in FIG. 6.

FIG. 6 presents a comparative curves diagram of the electrochemical signal response as recorded over time. As can be seen in FIG. 6, the recorded signals are not notable upon the addition of the substrates, glucose and PPD, marked by the left arrow in FIG. 6. The recorded signals are notable only after the addition of the α-dog-IgG-HRP conjugate, marked by the right arrow in FIG. 6.

These results clearly demonstrate the effectiveness of employing the bi-enzyme channeling effect to dog-IgG immunosensor electrodes, and since the reaction occurs on the surface of the working electrode, the signal is due to the specific binding events between the antigens and the corresponding antibodies (see, dark green and blue curves in FIG. 6 representing two experiments employing different electrode prepared and used under similar conditions). The ratio between the control experiments wherein BSA replaces the IgG (see, yellow and red curves in FIG. 6 representing two experiments employing different electrode prepared and used under similar conditions) is significant although activity signals are detected for the BSA-loaded electrodes, probably due to some levels of non-specific reactions, which can be reduced to a minimum with further optimization. In addition, the lack of a signal response at each time interval of substrate addition indicate that this systems and method are suitable for a “one-step” system wherein all the substrates and analytes are entered at once.

Successive Steps, No-Wash and Separation-Free Immunoassay Using Graphite Electrode and Acetaminophen as HRP Secondary Substrate:

The following experiment demonstrated the concept and use of enzyme channeling for “no-wash” immunoassays, using graphite (pencil lead) electrodes, artificial polymer PEI and HRP-substrate acetaminophen (AAP) as substrate, and further demonstrated the effect of the addition of the detergent Tween-20 as an attenuator of non-specific effects.

The use of acetaminophen, known commercially as paracetamol, as the HRP substrate instead of PPD was suggested so as to circumvent the use of the unstable and toxic PPD compound. Although acetaminophen is less sensitive as compared to PPD by factor of two, it is clearly none-toxic and can be safely used at the concentration administered in the assay. It is also less susceptible to light and thus can be kept as a stable powder in an immunosensor kit designed for commercial use.

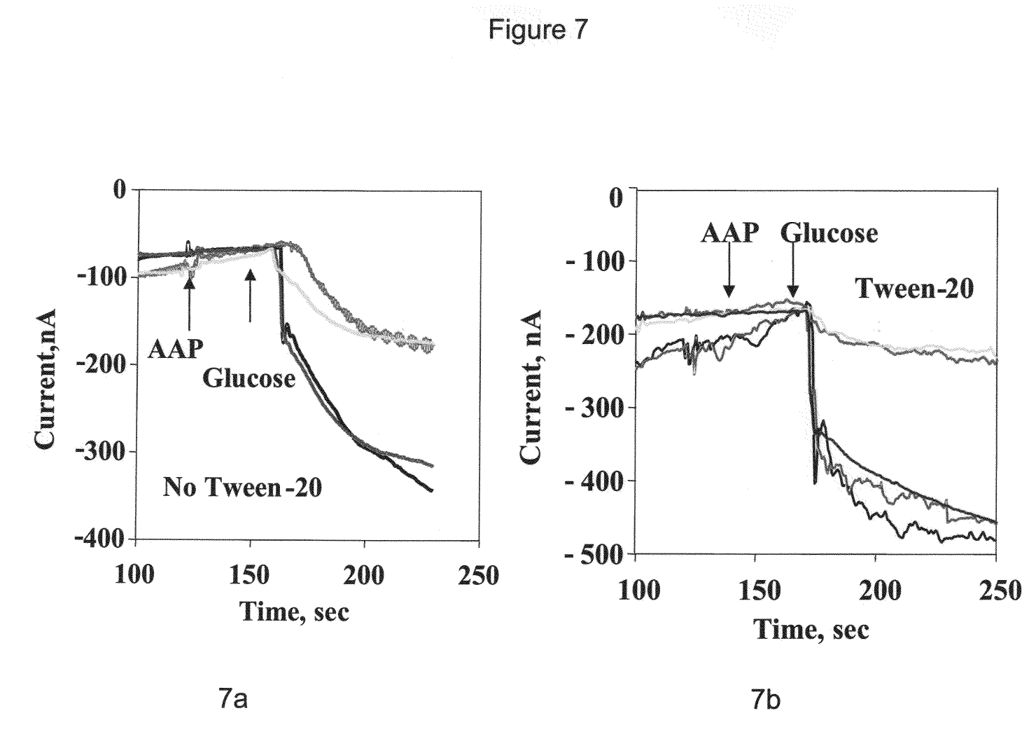

The experiments with acetaminophen (AAP) were performed as described hereinabove for PPD. These experiments were characterized by successive additions of the substrate AAP, followed by the addition of glucose, and finally the addition of the conjugate, α-dog-IgG-HRP (diluted 1:1000) tn 50 seconds intervals. Successive additions of the substrates and the conjugates without wash produced notable and systematic signals, which are presented in FIG. 7.

FIG. 7 presents two comparative curves diagrams of the electrochemical signal response as recorded over time. As can be seen in FIG. 7a, the notable signals were systematic and reproducible by duplicates. The addition of AAP did not affect the signal, nor the addition of glucose, and the difference between the experiments was noted only upon addition of the α-dog-IgG-HRP conjugate, differentiating between the tests conducted with immobilized dog-IgG (see, green and black curves in FIG. 7a) from the control tests conducted with immobilized BSA (see, red and yellow curves in FIG. 7a). Still, the non-specific signals recorded in the control experiments for the BSA-loaded electrodes are still significant.

As can be seen in FIG. 7b, similar and reproducible results were obtained while adding the detergent Tween-20 to the solution, yet the signal recorded upon addition of α-dog-IgG-HRP is 2-fold stronger as compared to the results of the experiments conducted without Tween-20 (see, green, blue and black curves in FIG. 7b). Furthermore, the non-specific signals recorded for the BSA-loaded electrodes (see, red and yellow curves in FIG. 7a) were considerably reduced by the addition of Tween-20, as compared to the results of the experiments conducted without Tween-20; hence a great improvement of the signal-to-noise ratio was achieved, especially when considering that the entire experiment was conducted without any wash steps.

These results also corroborate the assumption that this system too can be used in a “one-step” mode wherein all the substrates and analytes are entered at once.

One-Step, No-Wash and Separation-Free Immunoassay Using Graphite Electrode and Acetaminophen as HRP Secondary Substrate:

The following experiments intended to examine the response of the system to “one-step” addition of all substrates and the conjugate. Two sets of experiments were conducted showing that the ratio of the electrodes with the co-immobilized GOX and dog-IgG or BSA performed better at a ratio 1:1 of GOX (0.5 mg/ml) versus dog-IgG or BSA (0.5 mg/ml), as compared to the 1:2 ratio of GOX (0.5 mg/ml) versus dog-IgG or BSA (1 mg/ml).

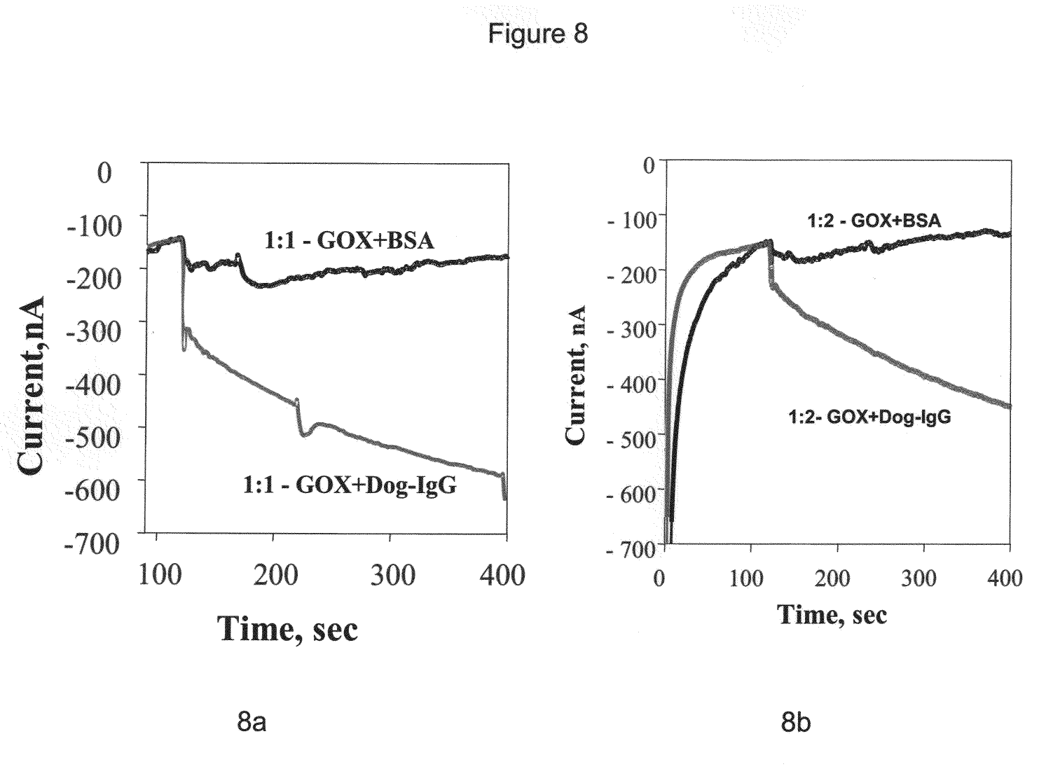

FIG. 8 presents two comparative curves diagrams of the electrochemical signal response as recorded over time. As can be seen in FIG. 8, the obtained results validated the “one-step” and separation-free approach, wherein all the components of the immunoassay and the bi-enzymatic reactions are co-added, by exhibiting signals which are notably high and reproducible for the dog-IgG electrode while the control experiments show no signal at all. The elimination of the none-specific interactions demonstrated the substantial improvement of the one-step approach.

As can be seen in FIG. 8a, showing the results obtained with the 1:1 co-immobilized electrode, adding all the components of the immunoassay and the bi-enzymatic reactions triggered the signal generation at once, and the signal-to-noise ratio between the dog-IgG-loaded electrode (see, red curve in FIG. 8a) and the BSA-loaded control electrode (see, black curve in FIG. 8a) kept improving with the prolongation of the measurement.

As can be seen in FIG. 8b, showing the results obtained with the 1:2 co-immobilized electrode, doubling the amount of immobilized dog-IgG and the BSA in the control experiment on the electrode did not improve the magnitude of the signal and even lowered the signal (see, red curve in FIG. 8b) as compared to the results obtained with the 1:1 co-immobilized electrode. Still, the signal recorded for the non-specific interactions was reduced (see, black curve in FIG. 8b) as compared to the results obtained with the 1:1 co-immobilized electrode.

One-Step and Separation-Free Measurements with the Avidin-Biotin Model Platform Using Graphite Electrode and Acetaminophen as HRP Secondary Substrate:

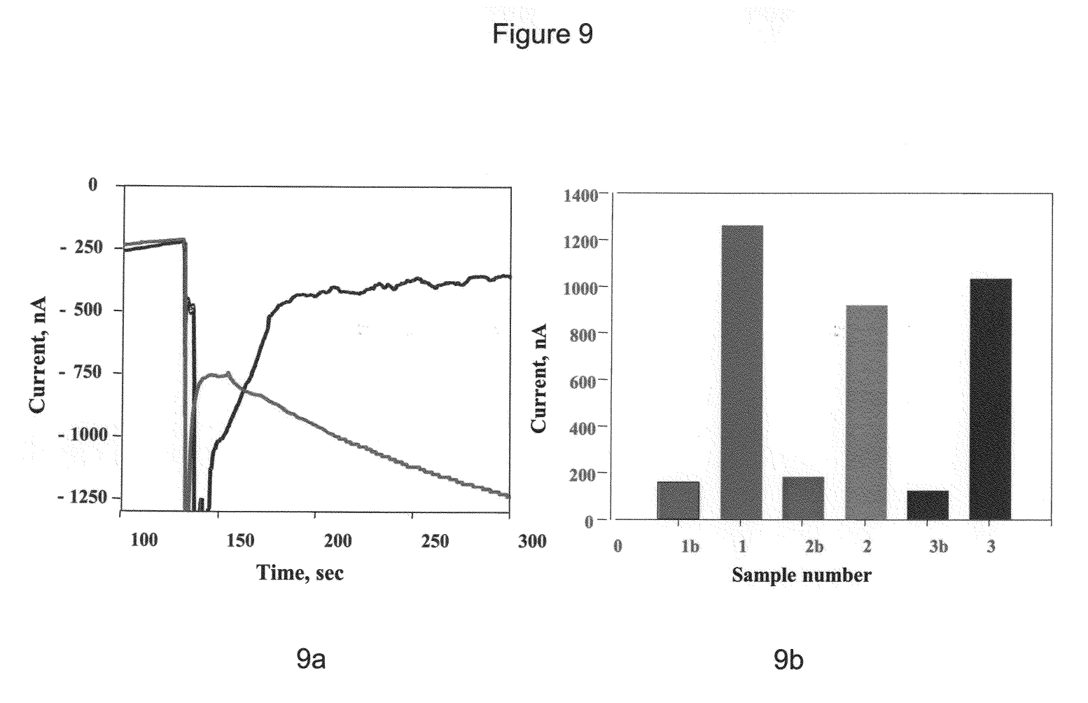

The avidin-biotin model platform was used to demonstrate the reliability of the basic concept of enzyme channeling in the context of the assays measured in the present immunosensor. Measurements were performed in one-step, separation free format without wash steps using a graphite electrode, as described hereinabove, by introducing the substrates and biotin-HRP to the electrodes. The results obtained with this platform are presented in FIG. 9.

FIG. 9a presents a comparative curves diagram of the electrochemical signal response as recorded over time. As can be seen in FIG. 9a, the reaction based on the avidin-biotin pair resulted in notable signals (see, red curve in FIG. 9a) while the control experiments showed no signal at all, and even an inversed signal (see, blue curve in FIG. 9a) with the prolongation of the measurement.

FIG. 9b presents a comparative bar diagram, comparing the maximal currents recorded for the electrochemical response of three repeating experiments, namely experiment 1 in red bars, experiment 2 in green bars and experiment 3 in blue bars, conducted with three different electrodes, wherein the currents obtained for the BSA-loaded electrodes, are represented by bars marked by the letter “b”, namely 1b, 2b and 3b, along side with the bars representing by the currents obtained by the avidin-loaded electrodes, namely 1, 2 and 3. As can be seen in FIG. 9b, this method is specific and reproducible, as demonstrated by the results of the three different repeats of the same experiment.

These results of the assay in this format clearly demonstrated the general reliability of the enzyme-channeling based immunoassay platform presented herein.

One-Step, No-Wash and Separation-Free Immunoassay Using SPEs and Acetaminophen as HRP Secondary Substrate:

The goal of these experiments was to show feasibility of the system with disposable three screen printed electrodes (SPEs) containing all three electrodes on one electrical circuit printed on one electrode plate, eliminating the need for a separated working electrode, such as the “pencil-lead” graphite electrode.

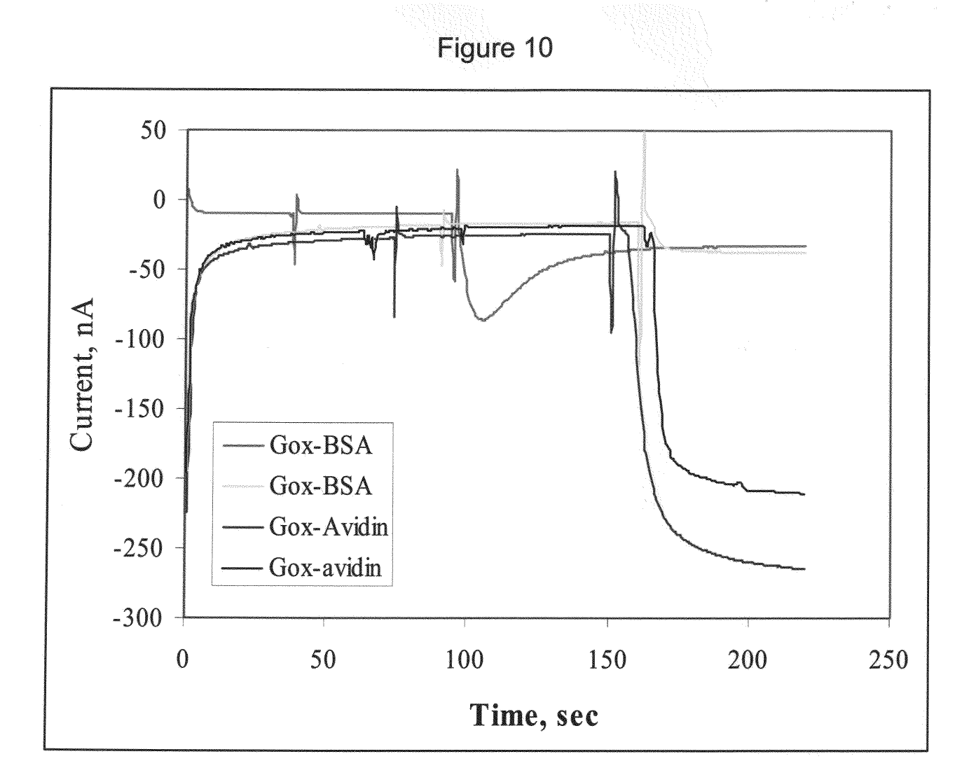

The experiments conducted with avidin-biotin platform as described hereinabove, in a one-step separation free format, and the duplicated results are presented in FIG. 10.

FIG. 10 presents a comparative curves diagram of the electrochemical signal response as recorded over time. As can be seen in FIG. 10, the notable signals produced by two repeating experiments using an avidin-loaded working SPE (see, black and blue curves in FIG. 10) were systematic and reproducible and exhibited high specificity in comparison to the two repeating control experiments using an BSA-loaded working SPE (see, red and yellow curves in FIG. 10). In addition, the measurements were in accordance with the results obtained from the well-characterized graphite electrodes presented hereinabove.

### Measurements Using Dog Sera

Measurements of Dog Sera Using Laminar Flow Membranes on SPEs:

The functionality of the Predator™ laminar flow membrane was first tested with intensive wash steps, without enzyme channeling and without laminar flow. Since the experiment was conducted without enzyme channeling, hydrogen peroxide was added to the measured reactions in the presence of acetaminophen.

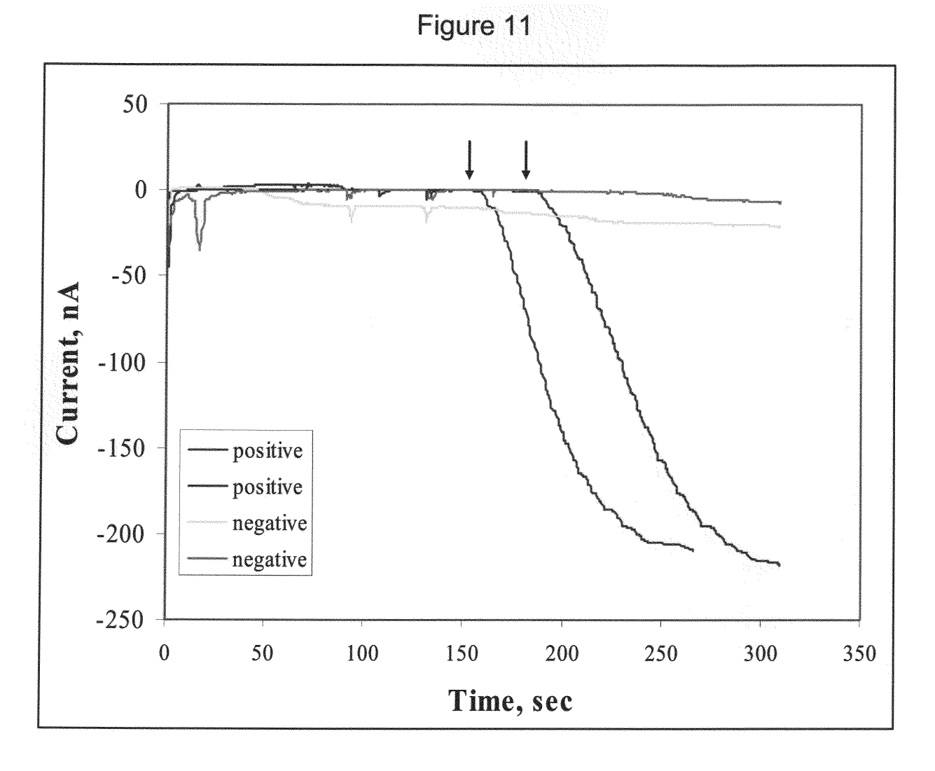

Distemper antigen (CDV) was covalently bound to the membrane, followed by blocking and incubation with positive or negative dog sera as described hereinabove. Thereafter the membranes were incubated with the α-dog-IgG-HRP conjugate, the membrane was laid on the SPE surface for the electrochemical measurements, and the signal produced in duplicates are presented in FIG. 11.

FIG. 11 presents a comparative curves diagram of the electrochemical signal response as recorded over time. As can be sees in FIG. 11, there are substantial differences between the minimal and noise-free signal recorded for the negative serum samples (see, red and yellow curves in FIG. 11) and the notable signal recorded for the positive serum samples (see, black and blue curves in FIG. 11). The signal response of the electrode commenced only upon the addition of hydrogen peroxide, indicated by the arrows (see, right arrow for the black curve, and the left arrow for the blue curve in FIG. 11), further demonstrating the stability and reliability of the immunoassay and the immunosensor presented herein, and further demonstrating the suitability of this type of membrane for SPE-based immunosensors.

Measurements of Dog Sera with Antigen-Loaded SPE:

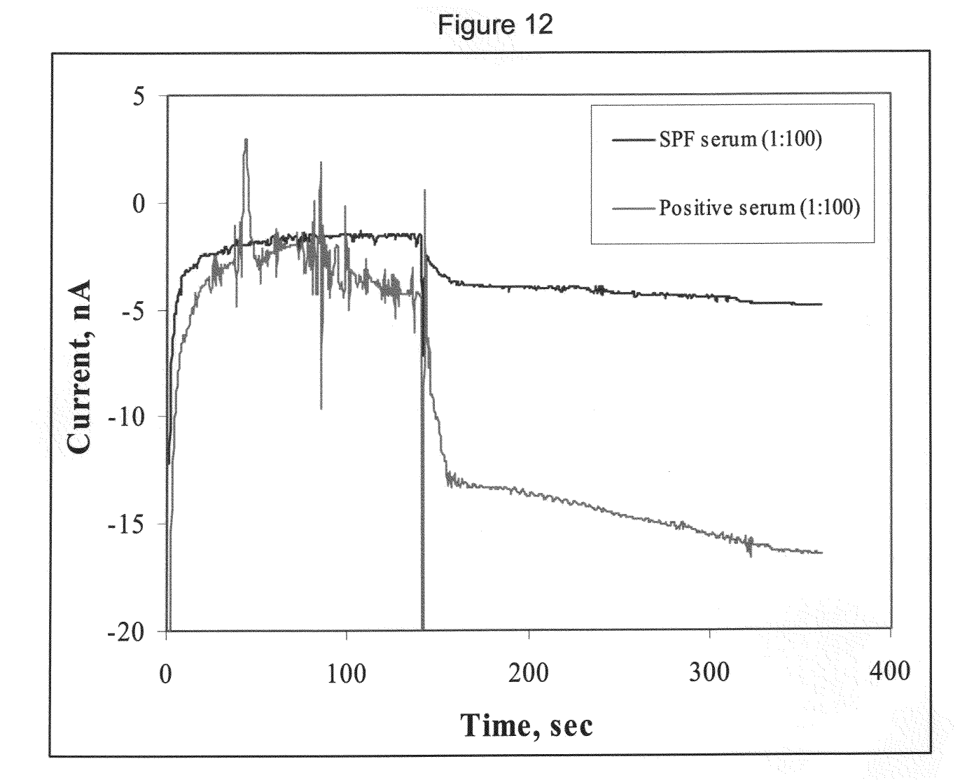

The ability of an SPE modified with PEI polymer and loaded with an antigen, to respond to dog sera using enzyme-channeling for signal generation and acetaminophen as HRP substrate was examined by immobilizing canine distemper virus antigen (CDV, diluted 1:20) via glutaraldehyde to the PEI polymer in the presence of GOX, followed by blocking with skim milk (1%), as described hereinabove. The signal from the electrode was measured without wash steps in the presence of positive or negative dog sera, and the results are presented in FIG. 12.

FIG. 12 presents a comparative curves diagram of the electrochemical signal response as recorded over time. As can be seen in FIG. 12, there is a notable signal for the positive serum (see, black curve in FIG. 12) and a slighter signal for the negative serum (SPF, see, magenta curve in FIG. 12), and in general the signals obtained were weaker and noisier than those obtained in the purified samples of antibodies or avidin-biotin experiments presented hereinabove.

Measurements of Dog Sera with Antigen-Loaded SPE Using Biotinylate Distemper Antigen:

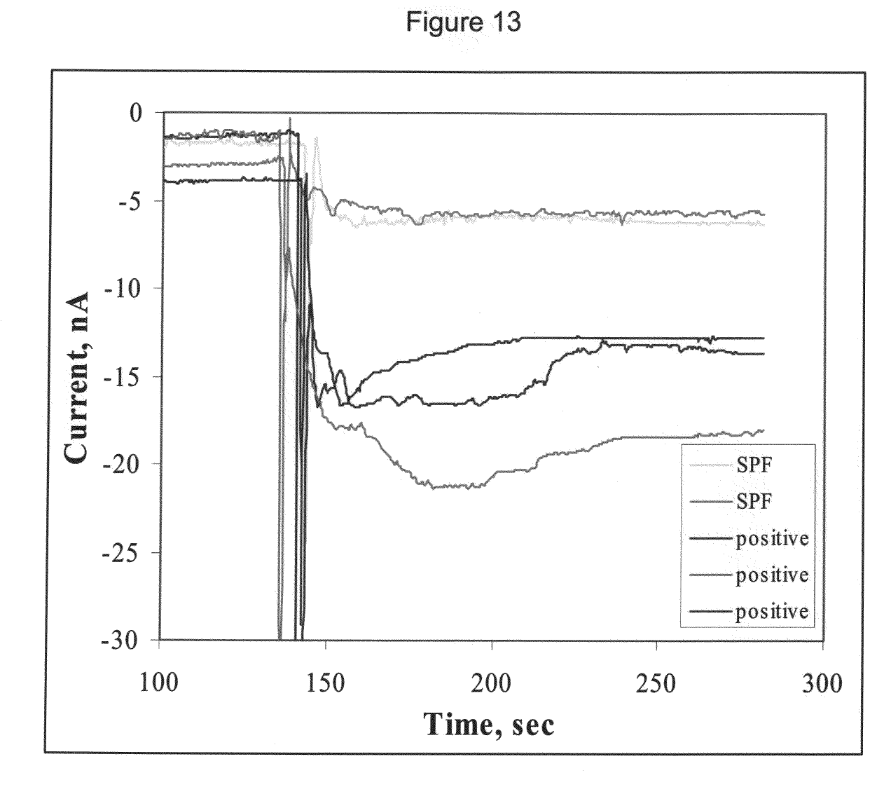

To improve the signal magnitude and the signal-to-nose ratio obtained from the antigen-loaded SPE as presented in the previous example, a biotinylate distemper antigen (biotin-CDV), prepared as described hereinabove, was used with avidin and GOX co-immobilized on a PEI modified SPE in order to make use of the avidin-biotin model platform described hereinabove. Avidin and GOX were co-immobilized to the PEI modified SPEs followed by binding with the biotin-CDV. The avidin/GOX electrodes were measured in the presence of positive and negative (SPF) serums, in the presence of α-dog-IgG-HRP, in separation-free sandwich format, and the results are presented in FIG. 13, showing the triplicated results for positive serum and duplicated results for SPF serum.

FIG. 13 presents a comparative curves diagram of the electrochemical signal response as recorded over time. As can be seen in FIG. 13, the notable signals recorded for the positive serum (see, black, blue and magenta curves in FIG. 13) correlated with the level of the antibodies for CDV in the sera while the signals recorded for the negative sera (SPF, see, red and yellow curves in FIG. 13) showed only a minimal signal, again with good correlation to the lack of antibodies for CDV in the sera.

Measurement of Different Serum Levels and Comparison with the Commercial ImmuniComb:

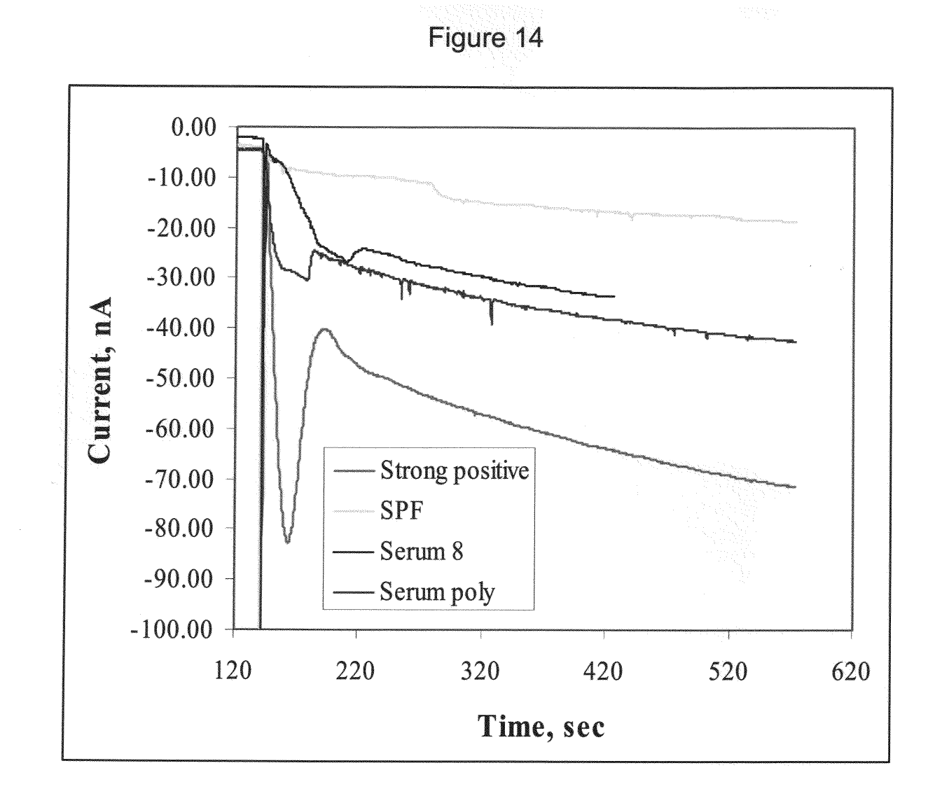

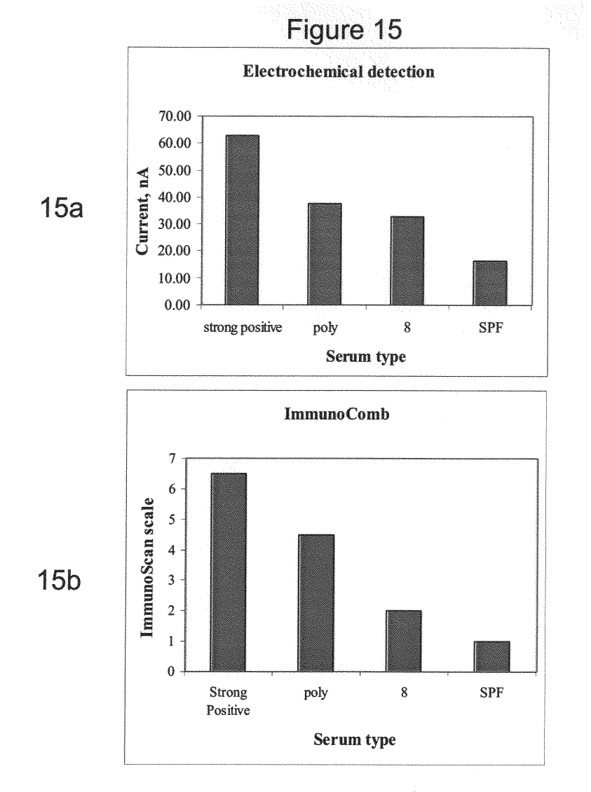

Avidin and GOX loaded electrodes were reacted with different dog sera which were known to have different titer levels of antibodies for CDV. The dog serum samples were denoted “strong positive”, “SPF” (negative), “serum 8” and “serum poly” as presented in FIG. 14. The same serum samples were measured with the commercial ImmunoComb and the results were compared, as presented in FIG. 15.

FIG. 14 presents a comparative curves diagram of the electrochemical signal response as recorded over time. As can be seen in FIG. 14, the “strong positive” serum sample (see, red curve in FIG. 14) generated the strongest signal, the “SPF” serum sample (see, yellow curve in FIG. 14) generated the weaker signal, the “serum 8” sample (see, black curve in FIG. 14) generated a slighter signal and the “poly” serum sample (see, blue curve in FIG. 14) generated a slightly stronger signal than the “serum 8” sample.

FIG. 15 presents two comparative bar diagrams, comparing the maximal currents recorded for the electrochemical response of the experiments presented in FIG. 14, namely the experiments conducted for the sera samples denoted “strong positive”, “SPF” (negative), “serum 8” and “serum poly”. As can be seen in FIGS. 14a and 14b, the electrochemical currents recorded using the “one-step, no-wash” enzyme-channeling immunoassays (FIG. 15a) exhibited high correlation to the results obtained using the commercial ImmunoComb assay kit (FIG. 15b). This correlation clearly demonstrates the reliability of the device and method presented herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-68. (canceled)

69. A system for detecting an antibody in a liquid sample, the system comprising:
an electrochemical cell which comprises:
a reference electrode;
a counter electrode;
an electrolytic solution;
a current detecting unit; and
a working electrode having immobilized thereon an antigen and a first enzyme of an enzymatic cascade;
a conjugate which comprises an agent capable of specifically binding to the antibody and a second enzyme of said enzymatic cascade being conjugated to said agent; and
a substrate of said first enzyme of said enzymatic cascade;
wherein:
said antigen is capable of specifically binding to the antibody; and
said first enzyme is capable of catalyzing a formation of a substrate of said second enzyme,
and further wherein said second enzyme generates an electrochemically detectable moiety upon binding of said conjugate to the antibody and binding of the antibody to the antigen,
whereas a presence and/or amount of said electrochemically detectable moiety is detectable by said detecting unit.

70. The system of claim 69, further comprising a secondary substrate of said second enzyme.

71. The system of claim 69, wherein said working electrode comprises an immobilization layer applied thereon.

72. The system of claim 69, wherein said antigen is not an antibody.

73. The system of claim 71, wherein said immobilization layer comprises a polymer attached to the surface of said working electrode and a cross-linking agent attached to said polymer.

74. The system of claim 73, wherein said immobilization layer comprises a microporous membrane.

75. A kit for detecting an antibody in a liquid sample, the kit comprising:
a working electrode having immobilized thereon an antigen and a first enzyme of an enzymatic cascade;
wherein:
said antigen is capable of specifically binding to the antibody;
said first enzyme is capable of catalyzing a formation of a substrate of a second enzyme; and
said second enzyme is capable of catalyzing a formation an electrochemically detectable moiety.

76. The kit of claim 75, further comprising:
a conjugate which comprises an agent capable of specifically binding to the antibody and said second enzyme of said enzymatic cascade being conjugated to said agent;
wherein:
said second enzyme generates said electrochemically detectable moiety upon binding of said conjugate to the antibody and binding of the antibody to said antigen.

77. The kit of claim 75, further comprising a substrate of said first enzyme of said enzymatic cascade.

78. The kit of claim 75, further comprising a secondary substrate of said second enzyme.

79. The kit of claim 75, wherein said antigen is not an antibody.

80. A method of detecting an antibody in a liquid sample, the method comprising:
contacting the liquid sample with the system of claim 69;
applying a pre-selected potential between said working electrode and said counter electrode;
recording a current formed between said working electrode and said counter electrode; and
determining said presence and/or amount of said electrochemically detectable moiety, thereby detecting the antibody in the liquid sample.

81. The method of claim 80, wherein said contacting is effected without washing said cell.

82. The method of claim 80, wherein said antigen is not an antibody.

83. A system for detecting a first member of a binding pair in a liquid sample, the system comprising:
an electrochemical cell which comprises:
a reference electrode;
a counter electrode;
an electrolytic solution;
a current detecting unit; and
a working electrode having immobilized thereon a second member of the binding pair and a first enzyme of an enzymatic cascade;
a conjugate which comprises an agent capable of specifically binding to the first member of the binding pair and a second enzyme of said enzymatic cascade conjugated to said agent;
a substrate of said first enzyme of said enzymatic cascade; and
a secondary substrate of said second enzyme of said enzymatic cascade, wherein:
said first enzyme of said enzymatic cascade is a hydrogen peroxide-producing enzyme;
said second enzyme of said enzymatic cascade is a peroxidase; and
said secondary substrate is acetaminophen,
and further wherein:
said second enzyme generates a detectable form of said acetaminophen upon binding of said conjugate to the first member of the binding pair and binding of the first member to said second member of the binding pair,
whereas a presence and/or amount of said detectable form of said acetaminophen is detectable by said detecting unit.

84. The system of claim 83, wherein said working electrode comprises an immobilization layer applied thereon.

85. A method of detecting a first member of a binding pair in a liquid sample, the method comprising:
contacting the liquid sample with the system of claim 83;
applying a pre-selected potential between said working electrode and said counter electrode;
recording a current formed between said working electrode and said counter electrode; and
determining said presence and/or amount of said detectable form of said acetaminophen, thereby detecting the first member of a binding pair in said liquid sample.

86. The method of claim 85, wherein said contacting is effected without washing said cell.

87. An electrode for detecting an antibody in a liquid sample, the electrode comprising a body and a surface having immobilized thereon an antigen and a first enzyme of an enzymatic cascade, said antigen is capable of specifically binding to the antibody, said first enzyme is capable of catalyzing a formation of a substrate of a second enzyme in said enzymatic cascade, said second enzyme capable of generating an electrochemically detectable moiety upon binding of a conjugate to the antibody and binding of the antibody to the antigen, whereby said conjugate comprises an agent capable of specifically binding to the antibody and said second enzyme of said enzymatic cascade being conjugated to said agent.

88. The electrode of claims 87, wherein said surface comprises an immobilization layer applied thereon.

89. The electrode of claim 87, wherein said antigen is not an antibody.

90. The electrode of claim 88, wherein said immobilization layer comprises a polymer attached to the surface of said working electrode and a cross-linking agent attached to said polymer.

91. The electrode of claim 90, wherein said immobilization layer comprises a microporous membrane.

92. The electrode of claim 87, wherein said second enzyme is a peroxidase.

93. The electrode of claim 92, wherein said secondary substrate is selected from the group consisting of potassium iodide (KI), p-phenylene diamine dihydrochloride (PPD) and acetaminophen.
